(19) United States
(12) Patent Application Publication
(10) Pub. No.: US 2017/0246260 A1
(43) Pub. Date: Aug. 31, 2017

(54) MODIFIED ANTIVIRAL NUCLEASE

(71) Applicant: Agenovir Corporation, South San Francisco, CA (US)

(72) Inventors: Derek Sloan, Belmont, CA (US); Stephen R. Quake, Stanford, CA (US)

(21) Appl. No.: 15/204,165

(22) Filed: Jul. 7, 2016

Related U.S. Application Data
(60) Provisional application No. 62/299,829, filed on Feb. 25, 2016.

(51) Int. Cl. A61K 38/46; C07K 14/195; C12N 9/96; A61K 47/48; C12N 9/22

(52) U.S. Cl. CPC A61K 38/465; A61K 47/48092; C12Y 301/00; C12N 9/22; C12N 9/96; C07K 14/195; C07K 2319/80; A61K 48/00

(57) ABSTRACT

A modified programmable nuclease provided as an antiviral therapeutic includes a programmable nuclease such as an RNA-guided nuclease, a DNA-guided nuclease, or a protein-guided nuclease linked to a secondary moiety to improve uptake, half-life, efficacy, or other properties. The nuclease is programmed to cleave viral genetic material in an infected patient.

### CROSS-REFERENCE TO RELATED APPLICATION

This application claims the benefit and priority of U.S. Provisional Application Ser. No. 62/299,829, filed Feb. 25, 2016, incorporated by reference.

### TECHNICAL FIELD

The invention relates to antiviral therapeutics.

### BACKGROUND

A person with a viral infection can experience significant discomfort, even severe pain or death. Rabies, chicken pox, the flu, shingles, hepatitis, and cancer are examples of painful or fatal conditions that may arise as a consequence of infection by a virus. Some of the most common targets of viral infection include the respiratory system, the gastrointestinal tract, the liver, the nervous system, and skin—all systems that are important to a healthy and productive life. Thus viral infections pose significant problems to human health and welfare.

A virus is an infectious agent that only replicates inside the cells of other living organisms. Typically, a virus will have its own genome with a limited number of genes, which include the genes for the virus's coat proteins. Some viral infections will provoke an immune response in the infected person that may eliminate the virus. However, some viruses cause chronic or latent infections that persist indefinitely.

Efforts have been made to develop drugs that target viral proteins but those efforts have not been wholly successful. For example, when a virus is in a latent state, not actively expressing its proteins, there is no protein to target. Additionally, viral therapeutics face other biological obstacles such as clearance of the therapeutic by the host immune system, poor uptake into infected cells, or activity levels that are less than desirable. As such, even though there are some viral therapeutics on the market, there are still many instances of people suffering from the consequences of viral infections.

### SUMMARY

The invention provides an antiviral therapeutic composition that includes a nuclease that is programmed to cleave viral genetic material and is also modified to improve therapeutic effect. Modifications of programmable nucleases of the invention may include linkage to a secondary moiety with a salutary effect on the composition. For example, linking the programmable nuclease with the Fc fragment of an immunoglobulin molecule (Fc-domain) or polyethylene glycol (PEG) may improve the biophysical and manufacturing properties by improving in vitro solubility, stability and/or purification, e.g., affinity purification of the Fc-domain with protein-G/A. In other embodiments, linking the programmable nuclease with a Fc-domain or PEG may improve the in vivo pharmacokinetic and/or pharmacodynamic properties by: reducing immunogenicity, promoting self-assembly into nanoparticles, facilitating tissue-specific delivery and cell penetration, e.g. Fc receptor-mediated uptake, and/or prolonging serum half-life. Linking a programmable nuclease to albumin or an elastin protein also may prolong the half-life of the therapeutic in the patient. In another example, a programmable nuclease may be linked to an apolipoprotein(E) (ApoE) protein to promote delivery across the blood-brain barrier and/or promote enhanced receptor-mediated cellular uptake. A programmable nuclease may be guided to a target infected cell by linkage to an aptamer, e.g., an aptamer that binds specifically to a viral protein. In other examples, a programmable nuclease may be linked to a cell-penetrating peptide to enhance cellular uptake. In other examples, a programmable nuclease is linked to biotin for ease of connecting to other features of interest, e.g., using avidin or streptavidin. Thus it can be seen that a programmable nuclease may be modified by linkage to an Fc-domain, albumin, biotin, PEG, aptamers, apoE, others, or combinations thereof.

Because the programmable nuclease is programmed to cleave viral genetic material, it is useful in an antiviral therapeutic. Because it is modified to improve manufacturing, tissue delivery, cellular uptake, immunogenicity, serum half-life, or another functionality, the composition has useful and beneficial properties in clinical applications. Thus, compositions of the invention are useful to treat patients with viral infections or diseases caused by viral infections, and to clear those infections or treat those virus-associated diseases, e.g., oncoviruses that cause and are associated with certain cancers. Since compositions of the invention target nucleic acid for cleavage, they may be used even where a viral infection is in a latent or persistent state. Thus the invention provides antiviral therapeutics that are programmed to target a particular viral infection, effective regardless of infection cycle stage, and optimized through specific modifications for usefulness as a therapeutic.

In certain aspects, the invention provides a composition for treating a viral infection. The composition includes a programmable nuclease linked to a secondary moiety. In certain embodiments, the programmable nuclease may be an RNA-guided nuclease (e.g., a CRISPR-associated nuclease, such as Cas9 or a modified Cas9 or Cpf1 or modified Cpf1). The programmable nuclease may be a TALEN or a modified TALEN. In certain embodiments, the programmable nuclease may be a DNA-guided nuclease (e.g., a Pyrococcus furiosus Argonaute (PfAgo) or Natronobacterium gregoryi Argonaute (NgAgo). The programmable nuclease may be connected to any suitable secondary moiety including, for example, polyethylene glycol (PEG), an aptamer, at least a portion of an apolipoprotein E (apoE) protein, an Fc region of an immunoglobulin, albumin, biotin, streptavidin, avidin, a lectin protein, a sugar, an elastin protein, a cell-penetrating peptide, an enzyme (such as a nuclease domain of FokI), a zinc finger protein, an antibody binding region, an enzyme cleavage region, a transcription repressor.

In some embodiments the secondary moiety is selected from the group consisting of polyethylene glycol (PEG); an aptamer; at least a portion of an apolipoprotein E (apoE) protein; at least a portion of an Fc region of an immunoglobulin; and at least a portion of albumin. The programmable nuclease may preferably be an RNA-guided nuclease (e.g., with an amino acid sequence at least 90% similar to Cas9). The nuclease may be present in ribonucleoprotein form with the nuclease complexed with a guide RNA, in which a portion of the guide RNA is complementary to a target in a viral genome and not substantially complementary to any part of a human genome. The secondary moiety is attached to the Cas9 at, for example, a side chain of an amino acid of the Cas9, wherein the side chain may present an amine, a carboxyl, a sulfhydryl, or a carbonyl. Optionally, the secondary moiety is attached to the side chain through a linker, which may include one or more of a disulfide bond, a thioether, an amine bond, a hydrazine linkage, an amide bond, an imidoester, a peptide bond, maleimide; polyethylene glycol (PEG), BM(PEG)n with 1<n<9, and biotin.

In certain embodiments, the nuclease and the secondary moiety are part of a fusion protein, e.g., expressed from a recombinant gene. The secondary moiety includes a polypeptide that may be at least a portion of an apolipoprotein E (apoE) protein, at least a portion of an Fc region of an immunoglobulin, at least a portion of albumin, biotin, others, or a combination thereof. Preferably the programmable nuclease is an RNA-guided nuclease. The secondary moiety and the RNA-guided nuclease may be part of a recombinant protein in the certain embodiments. The recombinant protein may include linker between the secondary moiety and the RNA-guided nuclease. The RNA-guided nuclease portion of the recombinant protein is preferably present in ribonucleoprotein form with the nuclease complexed with a guide RNA, in which a portion of the guide RNA is complementary to a target in a viral genome and not substantially complementary to any part of a human genome. The RNA-guided nuclease may be Cas9 or have an amino acid sequence at least 90% similar to Cas9. The linker may include a plurality of glycine residues.

In certain aspects, the invention provides a composition for treating a viral infection, the composition comprising a programmable nuclease linked to a secondary moiety, wherein the secondary moiety is linked to the programmable nuclease at a side chain of an amino acid of, an N-terminus of, or a C-terminus of the programmable nuclease. The programmable nuclease may be an RNA-guided nuclease and the secondary moiety may be, for example, polyethylene glycol (PEG); an aptamer; at least a portion of an apolipoprotein E (apoE) protein; at least a portion of an Fc region of an immunoglobulin (Fc-domain); or at least a portion of albumin.

In some embodiments, the programmable nuclease is present as ribonucleoprotein comprising an RNA-guided nuclease complexed with a guide RNA, wherein a portion of the guide RNA is complementary to a target in a viral genome and not substantially complementary to any part of a human genome. The RNA-guided nuclease may have an amino acid sequence at least 90% similar to Cas9.

The secondary moiety may be attached to the side chain through a linker, which may include, for example, one or any combination of a disulfide bond; a thioether; an amine bond; a hydrazine linkage; an amide bond; an imidoester; a peptide bond; maleimide; a click reaction product; one or more five-membered heterocycles; polyethylene glycol (PEG); BM(PEG)n with 1<n<9; poly lactic-co-glycolic acid (PLGA)-b-PEG; and biotin.

In certain embodiments, the programmable nuclease is present as deoxyribonucleoprotein (DNP). For example, the DNP may comprise an NgAgo protein in complex with guide DNA (gDNA) that is complementary to a target in a viral genome and not substantially complementary to any part of a human genome, and the secondary moiety may be selected from the group consisting of polyethylene glycol (PEG); an aptamer; at least a portion of an apolipoprotein E (apoE) protein; at least a portion of an Fc region of an immunoglobulin (Fc-domain); and at least a portion of albumin. The DNA-guided nuclease may be NgAgo or a modified NgAgo having an amino acid sequence at least 90% similar to NgAgo.

In embodiments, the programmable nuclease comprises a TALEN protein engineered to recognize a target in a viral genome but not recognize the human genome. The secondary moiety may be, for example, polyethylene glycol (PEG); an aptamer; at least a portion of an apolipoprotein E (apoE) protein; at least a portion of an Fc region of an immunoglobulin (Fc-domain); at least a portion of albumin; or combinations thereof.

In some aspects, the invention provides a composition for treating a viral infection, in which the composition includes a programmable nuclease linked to a secondary moiety with the secondary moiety and the programmable nuclease both being part of a recombinant protein. The programmable nuclease may be a TALEN protein engineered to recognize a target in a viral genome but not recognize the human genome; a DNA-guided nuclease; or an RNA-guided nuclease. The secondary moiety may be at least a portion of an apolipoprotein E (apoE) protein; at least a portion of an Fc region of an immunoglobulin; at least a portion of albumin; biotin; or combinations thereof. The recombinant protein may include a linker between the secondary moiety and the programmable nuclease.

In certain embodiments, the programmable nuclease portion of the recombinant protein is an RNA-guided nuclease and the RNA-guided nuclease is present in ribonucleoprotein form with the nuclease complexed with a guide RNA, wherein a portion of the guide RNA is complementary to a target in a viral genome and not substantially complementary to any part of a human genome. In particular, the secondary moiety may be selected from the group consisting of at least a portion of an apolipoprotein E (apoE) protein; at least a portion of an Fc region of an immunoglobulin; at least a portion of albumin; and biotin. The RNA-guided nuclease portion of the recombinant protein may be Cas9 or a modified Cas9 having an amino acid sequence at least 90% similar to Cas9.

Any suitable linker may be included in the recombinant protein. For example, the linker may include a plurality of glycine residues.

Aspects of the invention provide a method for treating a viral infection. The method includes administering, to a patient with a viral infection, a programmable nuclease linked to a secondary moiety, wherein the secondary moiety is linked to the programmable nuclease at a side chain of an amino acid of the programmable nuclease. The secondary moiety may include, for example, one or any combination of polyethylene glycol (PEG); an aptamer; at least a portion of an apolipoprotein E (apoE) protein; at least a portion of an Fc region of an immunoglobulin (Fc-domain); and at least a portion of albumin.

In some embodiments, the programmable nuclease is an RNA-guided nuclease and The programmable nuclease may be administered in the form of a ribonucleoprotein (RNP) in which the RNA-guided nuclease is complexed with a guide RNA, in which a portion of the guide RNA is complementary to a target in a viral genome and not substantially complementary to any part of the patient's genome. The method may include the use of a modified programmable nuclease such as an RNA-guided nuclease with an amino acid sequence at least 90% similar to Cas9.

In certain embodiments, the programmable nuclease a DNA-guided nuclease and is administered in form of a deoxyribonucleoprotein (DNP). For example, the DNP may include an NgAgo protein in complex with guide DNA (gDNA) that is complementary to a target in a viral genome and not substantially complementary to any part of the patient's genome. A modified DNA-guided nuclease may be used such as one having an amino acid sequence at least 90% similar to NgAgo.

Embodiments of the method include the administration of a TALEN protein engineered to recognize a target in a viral genome but not recognize the patient's genome.

The secondary moiety is attached to the side chain through a linker. The linker may include one or more of a disulfide bond; a thioether; an amine bond; a hydrazine linkage; an amide bond; an imidoester; a peptide bond; maleimide; a click reaction product; one or more five-membered heterocycles; polyethylene glycol (PEG); BM(PEG)n with 1<n<9; poly lactic-co-glycolic acid (PLGA)-b-PEG; and biotin.

Aspects of the invention provide a method for treating a viral infection in which the method includes administering to a patient with the viral infection a composition comprising a programmable nuclease linked to a secondary moiety, wherein the secondary moiety and the programmable nuclease are both part of a recombinant protein. The programmable nuclease may be, for example, a TALEN protein engineered to recognize a target in a viral genome but not recognize the human genome; a DNA-guided nuclease; and an RNA-guided nuclease. The secondary moiety may include one or a combination of an apolipoprotein E (apoE) protein; at least a portion of an Fc region of an immunoglobulin; at least a portion of albumin; and biotin. The recombinant protein may include a linker between the secondary moiety and the programmable nuclease.

In other aspects, the invention provides a method of making a composition for treating a viral infection. The composition includes a programmable nuclease linked to a secondary moiety.

The composition may be made by linking the secondary moiety is linked to the programmable nuclease, for example, at a side chain of an amino acid, an N-terminus, or a C-terminus of the programmable nuclease. The programmable nuclease may be an RNA-guided nuclease such as Cas9 or Cpf1; a modified RNA-guided nuclease, e.g., with an amino acid sequence at least 90% similar to Cas9 or Cpf1; a DNA-guided nuclease such as NgAgo; a modified DNA-guided nuclease, e.g., with an amino acid sequence at least 90% similar to NgAgo; or a TALEN protein engineered to recognize a target in a viral genome but not recognize the patient's genome. The method may include linking the programmable nuclease to secondary moiety such as one or a combination of polyethylene glycol (PEG); an aptamer; at least a portion of an apolipoprotein E (apoE) protein; at least a portion of an Fc region of an immunoglobulin (Fc-domain); and at least a portion of albumin. The linking may include the use of chemical linkers and reagents, click chemistry, or other chemical methods. By such means, the secondary moiety may be attached to a side chain through a linker that may include one or more of a disulfide bond; a thioether; an amine bond; a hydrazine linkage; an amide bond; an imidoester; a peptide bond; maleimide; a click reaction product; one or more five-membered heterocycles; polyethylene glycol (PEG); BM(PEG)n with 1<n<9; poly lactic-co-glycolic acid (PLGA)-b-PEG; and biotin.

The composition may be made by providing a recombinant gene that encodes a recombinant fusion protein in which the programmable nuclease is linked to the secondary moiety. The gene may be provided as part of a vector such as a plasmid or an adeno-associated virus and the protein may be expressed in culture, e.g., within E. coli, a Lactobacillus, yeast, or other such organism. Alternatively, the recombinant gene may be delivered to the patient (e.g., as a plasmid in liposome or other nanoparticle) to be expressed in the patient's cells. The programmable nuclease may be a TALEN protein engineered to recognize a target in a viral genome but not recognize the human genome; a DNA-guided nuclease; and an RNA-guided nuclease; or a modified DNA-guided or RNA-guided nuclease. Preferably, the secondary moiety includes one or more of at least a portion of an apolipoprotein E (apoE) protein; at least a portion of an Fc region of an immunoglobulin; at least a portion of albumin; and biotin. The recombinant protein may include a linker between the secondary moiety and the programmable nuclease (e.g., a plurality of glycine residues or any of the other proteinaceous linkers shown herein).

Various features and embodiments are within the scope of the invention. The foregoing aspects or embodiments may variously include any of the following features or structures.

The secondary moiety may be non-covalently bound to the programmable nuclease (e.g., through a biotin/streptavidin linkage). The nuclease may be covalently linked to the secondary moiety. The nuclease may be linked to the secondary moiety through a linker (e.g., a protein linker that includes a plurality of proline residues, a plurality of glycine residues, a plurality of threonine and serine residues, or combinations thereof). The linker may be a non-protein chemical linker (e.g., based on a carbonyl, a disulfide bond, a thioether, an amine bond, a hydrazine linkage, an amide bond, an imidoester or click chemistries, such as cycloadditions of azides and alkynes to form heterocyclic structures or derivatives thereof). In some embodiments, the linker comprises polyethylene glycol (PEG) (e.g., formed with BM(PEG)n or BS(PEG)n with 1<n<9) or poly lactic-co-glycolic acid (PLGA)-b-PEG. In certain embodiments, the linker is attached to the programmable nuclease at an amino acid with a side chain comprising an amine, a carboxyl, a sulfhydryl, or a carbonyl (e.g., at an amino acid such as lysine, cysteine, aspartic acid, or glutamic acid). The linker may be flexible, rigid, biodegradable, cleavable or exhibit other useful properties. For example, the linker may include an enzyme cleavage region, e.g., a target region for a protease.

In some embodiments, the nuclease comprises at least one mutation such as an insertion or deletion (indel) or a substitution relative to a naturally-occurring version of the nuclease. For example, one or more substitutions may be included to reduce intracellular protein binding of the nuclease or reduce off-target toxicity of the composition. In other embodiments, the secondary moiety is at least a portion of an antibody non-covalently bound to the programmable nuclease. The secondary moiety may be an aptamer non-covalently bound to the programmable nuclease. Other variations are within the scope of the invention.

In the various embodiments, virus selected from the group consisting of a human papilloma virus (HPV), Epstein-Barr virus (EBV), cytomegalovirus (CMV), herpes simplex virus (HSV), hepatitis B virus (HBV) and Merkel cell polyomavirus (MCV).

In certain embodiments, compositions include, as the modified programmable nuclease, an RNA-guided nuclease (e.g., Cas9) and at least one gRNA targeting the genome of the virus. Suitable targets in viral genomes include, but are not limited to, a portion of a genome or gene of adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, Junin virus, Lassa virus, machupo virus, sabia virus, Crimean-Congo hemorrhagic fever virus, Ebola virus, Marburg virus, measles virus, mumps virus, parainfluenza virus, respiratory syncytial virus, human metapnemovirus, Hendra virus, Nipah virus, rabies virus, hepatitis D virus, rotavirus, orbivirus, Coltivirus, or Banna virus. In preferred embodiments, compositions of the invention are provided as antiviral therapeutics that include a modified programmable nuclease programmed to treat an infection by a hepatitis virus, a hepatitis B virus (HBV), an Epstein-Barr virus, a Kaposi's sarcoma-associated herpesvirus (KSHV), a herpes-simplex virus (HSV), a cytomegalovirus (CMV), human papilloma virus (HPV), and Merkel cell polyomavirus. The modified programmable nuclease is programmed (e.g., by a gRNA) to bind to or cleave a particular target.

The target in the viral genome may lie within one or more of a preC promoter in a hepatitis B virus (HBV) genome, an S1 promoter in the HBV genome, an S2 promoter in the HBV genome, an X promoter in the HBV genome, a viral Cp (C promoter) in an Epstein-Barr virus genome, a minor transcript promoter region in a Kaposi's sarcoma-associated herpesvirus (KSHV) genome, a major transcript promoter in the KSHV genome, an Egr-1 promoter from a herpes-simplex virus (HSV), an ICP 4 promoter from HSV-1, an ICP 10 promoter from HSV-2, a cytomegalovirus (CMV) early enhancer element, a cytomegalovirus immediate-early promoter, an HPV early promoter, and an HPV late promoter. In an exemplary embodiment, the virus is hepatitis B and the gRNA includes one or more of sgHBV-RT, sgHBV-Hbx, sgHBV-Core, and sg-HBV-PerS1.

In a preferred embodiment, the modified programmable nuclease is obtained or delivered in a ribonucleoprotein (RNP) form, e.g., as synthetic guide RNA (gRNA) complexed with a recombinant Cas9 protein chemically linked or fused to a secondary moiety described herein. It may be found that delivery as RNP is more effective than delivery via a nucleic acid vector that encodes the modified programmable nuclease, and that RNP permits delivery of pre-formed enzymatically active drug (which acts faster), and is only active in the cell for a very limited time (<24 hours), thus reducing non-specific toxicity and off-target activity. RNP can be directly electroporated into primary tissues, e.g. peripheral blood mononuclear cells (PBMCs), for ex vivo transplant indications. In another embodiment, the modified programmable nuclease is obtained or delivered as an mRNA that encodes the nuclease and is delivered with a gRNA. Compositions of the invention (e.g., proteins, RNP, gRNA and mRNA, or pDNA) may be incorporated into cationic lipid nanoparticles, solid liponanoparticles, liposomes, polymers or other formulations for in vivo delivery indications, e.g. cancer.

### BRIEF DESCRIPTION OF THE DRAWINGS

FIG. 1 shows a composition for treating a viral infection.

FIG. 2 depicts the amino acid sequence of Cas9.

FIG. 3 diagrams a programmable nuclease linked to an aptamer.

FIG. 4 diagrams a programmable nuclease linked to apoE.

FIG. 5 diagrams a programmable nuclease linked to an Fc region.

FIG. 6 diagrams a programmable nuclease linked to albumin.

FIG. 7 shows a programmable nuclease and a secondary moiety in a recombinant protein.

FIG. 8 diagrams a primary structure of a recombinant protein.

FIG. 9 shows a nucleic acid vector encoding a modified programmable nuclease.

FIG. 10 diagrams a composition with a programmable nuclease linked to albumin.

FIG. 11 shows a programmable nuclease linked to biotin.

FIG. 12 diagrams a pathway for attaching a secondary moiety at a carbonyl.

FIG. 13 diagrams a pathway for attaching a secondary moiety at a primary amine.

FIG. 14 shows a pathway for attaching a secondary linker via a thioether bond.

FIG. 15 shows a pathway for linking a secondary moiety via a disulfide bond.

FIG. 16 shows a pathway for biotinylating a primary amine.

FIG. 17 illustrates the reaction of an imidoester crosslinker with a primary amine.

FIG. 18 shows the use of BS(PEG)2 to link to an amine of a programmable nuclease.

FIG. 19 charts steps of a method of preparing an antiviral compositions.

FIG. 20 shows preparation of an antiviral composition.

FIG. 21 shows a programmable nuclease conjugated to a lectin protein.

FIG. 22 shows a composition with a programmable nuclease linked to a carbohydrate.

FIG. 23 shows the primary structure of a programmable nuclease linked to elastin.

FIG. 24 shows a programmable nuclease linked to a cell penetrating peptide.

FIG. 25 shows primary structure of a programmable nuclease connected to an enzyme

FIG. 26 shows a composition with a programmable nuclease linked to a zinc finger.

### DETAILED DESCRIPTION

Embodiments of the invention relate to the modification of programmable nucleases to optimize antiviral therapeutic qualities of those nucleases.

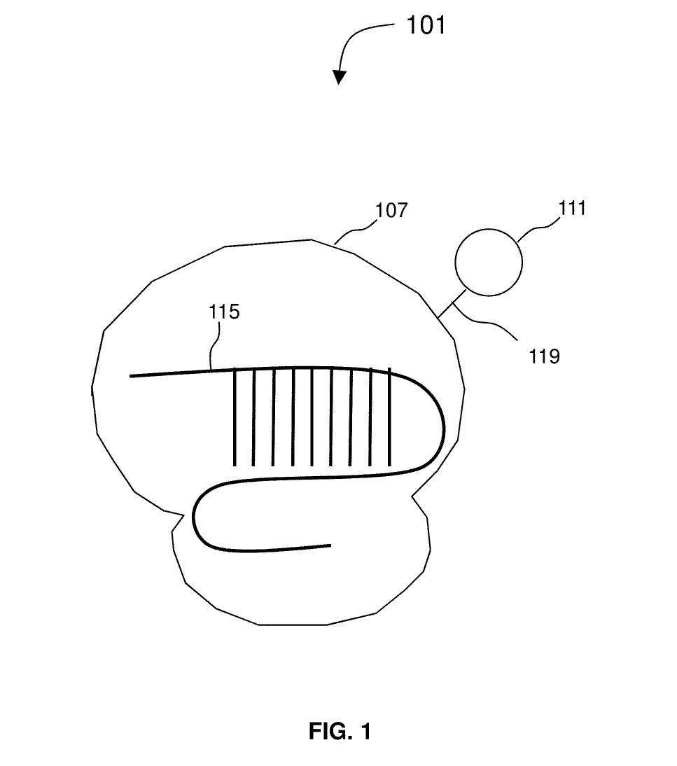

FIG. 1 shows a composition 101 for treating a viral infection. The composition includes a programmable nuclease 107 linked to a secondary moiety 111. The secondary moiety 111 is attached to the programmable nuclease 107 at a side chain of an amino acid of the nuclease. This example shows the secondary moiety being polyethylene glycol (PEG). PEGylation is one way to provide a programmable nuclease linked to a secondary moiety, PEG. Known PEGylation reagents may react with the amino groups of lysine residues (and to a minor degree with other protein nucleophiles: N-terminal amino groups, the imidazolyl nitrogen of histidine residues, as well as the side chains of serine, threonine, tyrosine and cysteine residues).

Optionally, a linker 119 extends between the programmable nuclease 107 and the secondary moiety 111. Any suitable programmable nuclease may be used including, for example, Cas9, ZFNs, TALENs, Cpf1, NgAgo, or a modified programmable nuclease having an amino acid sequence substantially similar to the unmodified version, for example, a programmable nuclease having an amino acid sequence at least 90% similar to one of Cas9, ZFNs, TALENs, Cpf1, or NgAgo, or any other programmable nuclease.

Programmable nuclease generally refers to an enzyme that cleaves nucleic acid that can be or has been designed or engineered by human contribution so that the enzyme targets or cleaves the nucleic acid in a sequence-specific manner. Programmable nucleases include zinc-finger nucleases (ZFNs), transcription activator-like effector nucleases (TALENs) and RNA-guided nucleases such as the bacterial clustered regularly interspaced short palindromic repeat (CRISPR)-Cas (CRISPR-associated) nucleases or Cpf1. Programmable nucleases also include PfAgo and NgAgo.

ZFNs cut genetic material in a sequence-specific matter and can be designed, or programmed, to target specific viral targets. A ZFN is composed of two domains: a DNA-binding zinc-finger protein linked to the FokI nuclease domain. The DNA-binding zinc-finger protein is fused with the non-specific FokI cleave domain to create ZFNs. The protein will typically dimerize for activity. Two ZFN monomers form an active nuclease; each monomer binds to adjacent half-sites on the target. The sequence specificity of ZFNs is determined by ZFPs. Each zinc-finger recognizes a 3-bp DNA sequence, and 3-6 zinc-fingers are used to generate a single ZFN subunit that binds to DNA sequences of 9-18 bp. The DNA-binding specificities of zinc-fingers is altered by mutagenesis. New ZFPs are programmed by modular assembly of pre-characterized zinc fingers.

Transcription activator-like effector nucleases (TALENs) cut genetic material in a sequence-specific matter and can be designed, or programmed, to target specific viral targets. TALENs contain the FokI nuclease domain at their carboxyl termini and a class of DNA binding domains known as transcription activator-like effectors (TALEs). TALEs are composed of tandem arrays of 33-35 amino acid repeats, each of which recognizes a single base-pair in the major groove of target viral DNA. The nucleotide specificity of a domain comes from the two amino acids at positions 12 and 13 where Asn-Asn, Asn-Ile, His-Asp and Asn-Gly recognize guanine, adenine, cytosine and thymine, respectively. That pattern allows one to program TALENs to target viral nucleic acid.

RNA-guided nucleases were first found as part of bacterial immune systems. The host bacteria capture small DNA fragments (˜20 bp) from invading viruses and insert those sequences (termed protospacers) into their own genome to form a CRISPR. Those CRISPR regions are transcribed as pre-CRISPR RNA(pre-crRNA) and processed to give rise to target-specific crRNA. Invariable target-independent trans-activating crRNA (tracrRNA) is also transcribed from the locus and contributes to the processing of precrRNA. The crRNA and tracrRNA have been shown to be combinable into a single guide RNA. As used herein, “guide RNA” or gRNA refers to either format. The gRNA forms a RNP with Cas9, and the RNP cleaves a target that includes a portion complementary to the guide sequence in the gRNA and a sequence known as proto spacer adjacent motif (PAM). The RNA-guided nucleases are programmed to target a specific viral nucleic acid by providing a gRNA that includes a ˜20-bp guide sequences that is substantially complementary to a target in viral nucleic acid. The targetable sequences include, among others, 5″-X 20NGG-3″ or 5″-X 20NAG-3″; where X 20 corresponds to the 20-bp crRNA sequence and NGG and NAG are PAMs. It will be appreciated that recognition sequences with lengths other than 20 bp and PAMs other than NGG and NAG are known and are included within the scope of the invention.

Argonaute proteins are a family of proteins that play a role in RNA silencing as a component of the RNA-induced silencing complex (RISC). The Argonaute of the archaeon Pyrococcus furiosus (PfAgo) uses small 5′-phosphorylated DNA guides to cleave both single stranded and double stranded DNA targets, and does not utilize RNA as guide or target.

NgAgo uses 5′ phosphorylated DNA guides (so called “gDNAs”) and appear to exhibit little preference for any certain guide sequences and thus may offer a general-purpose DNA-guided programmable nuclease. NgAgo does not require a PAM sequence, which contributes to flexibility in choosing a genomic target. NgAgo also appears to outperform Cas9 in GC-rich regions. NgAgo is only 887 amino acids in length. NgAgo randomly removes 1-20 nucleotides from the cleavage site specified by the gDNA. Thus, PfAgo and NgAgo represent potential DNA-guided programmable nucleases that may be modified for use as a composition of the invention.

In FIG. 1, the secondary moiety is PEG. PEGylation may aid in avoiding immune detection. Known PEGylation reagents react with the amino groups of lysine residues (and to a minor degree with other protein nucleophiles: N-terminal amino groups, the imidazolyl nitrogen of histidine residues, as well as the side chains of serine, threonine, tyrosine and cysteine residues). Many existing PEGylation reagents possess an activated carbonyl group in the form of N-hydroxy-succinimide esters that form stable protein-PEG conjugates via amide linkages. Thus, in some embodiments, the invention provides PEGylated programmable nuclease.

More generally, compositions of the invention may use modification of a programmable nuclease to reduce immunogenicity by eliminating/reducing potential T and B cell epitopes from the nuclease. Computer algorithms or in vitro assays may be used to map the locations of such epitopes within proteins (e.g., within a programmable nuclease or a proteinaceous secondary moiety). For example, the EpiMatrix suite of computational tools, together with ex vivo immunogenicity testing, may be applied to evaluate a composition of the invention to predict immunogenicity. Where T and B cell epitopes are discovered on a programmable nuclease, the nuclease may be modified to reduce or eliminate those epitopes. Such modification may include introduction one or more amino acid substitutions within the epitopes. Additionally or alternatively, an epitope may be blocked by linking a secondary moiety to the protein to prevent access to the epitope by an immune cell. Additionally, without being bound by any mechanism, it may be found that promiscuous or other modification of a protein decreases immunogenicity by prevent access to such epitopes. For example, linking an Fc-domain or PEGylating (e.g., at primary amines or carboxylic acid side chains) or attaching any other suitable secondary moiety may reduce or eliminate effective B and T cell epitopes available at a surface of the composition. Thus in some embodiments, the invention provides a composition for treating a viral infection, in which the composition includes a programmable nuclease linked to a secondary moiety, such that the programmable nuclease has decreased immunogenicity relative to an unmodified version thereof, due to the reduction or elimination of epitopes available to immune cells. As shown in FIG. 1, the composition 101 includes a Cas9 ribonucleoprotein complex (Cas9+gRNA) with PEG 111 linked at a plurality of locations that include the side chains of lysing residues (e.g., K3, K4, K26, K30, K31, K33, K44, K45, K65, K76, K92, K111, K112, K131, K140, K141, K148, K163, K183, K209, K218, K233, K234, K253, K263, K268, K294, K314, K323, K336, K346, K348, K374, K377, K382, K392, K401, K434, K439, K442, K468, K484, K500, K506, K510, K526, K528, K536, K545, K546, K554, K558, K562, K565, K570, K571, K599, K602, K604, K637, K649, K652, K665, K673, K677, K684, K705, K710, K734, K735, K742, K749, K755, K772, K775, K782, K789, K797, K810, K848, K855, K862, K866, K877, K878, K880, K890, K896, K902, K913, K918, K929, K942, K948, K954, K959, K961, K968, K974, K999, K1000, K1014, K1020, K1024, K1031, K1035, K1047, K1059, K1076, K1085, K1096, K1097, K1107, K1113, K1118, K1123, K1124, K1129, K1130, K1148, K1151, K1153, K1155, K1156, K1158, K1161, K1176, K1185, K1188, K1191, K1192, K1197, K1200, K1211, K1222, K1231, K1244, K1246, K1255, K1263, K1278, K1289, K1296, K1300, K1325, K1334, and K1340).

In the embodiment illustrated by FIG. 1, the RNA-guided nuclease is present as a ribonucleoprotein that includes Cas9 complexed with a guide RNA 115. A portion of the guide RNA 115 is complementary to a target in a viral genome and not substantially complementary to any part of a human genome.

The invention includes modifications to the programmable nuclease 107 to optimize antiviral therapeutic properties of the programmable nuclease. In certain embodiments, the programmable nuclease is an RNA-guided nuclease such as Cas9 or Cpf1.

Modifications to the programmable nuclease 107 may improve function. Certain modifications (e.g., PEGylation, fusion, aptamers, ApoE, Fc, albumin, elastin, cell-penetrating peptides) may improve immunogenicity, cell penetration, serum half-life, tissue targeting, therapeutic index, and nuclear localization. Additionally or alternatively, a programmable nuclease may include mutations such as indels or amino acid substitutions relative to wild type that reduce intracellular protein binding and off-target toxicity including reduction of toxicity attributable to nuclease expression or presence in cells that do not contain virus.

### i. MODIFIED SIDE CHAINS/FUNCTIONAL GROUPS

Embodiments of the invention provide a composition for treating a viral infection that includes a programmable nuclease linked to a secondary moiety via a linkage wherein one of the programmable nuclease is linked to the other through a side chain, N-terminus, or C-terminus of an amino acid in a peptide sequence.

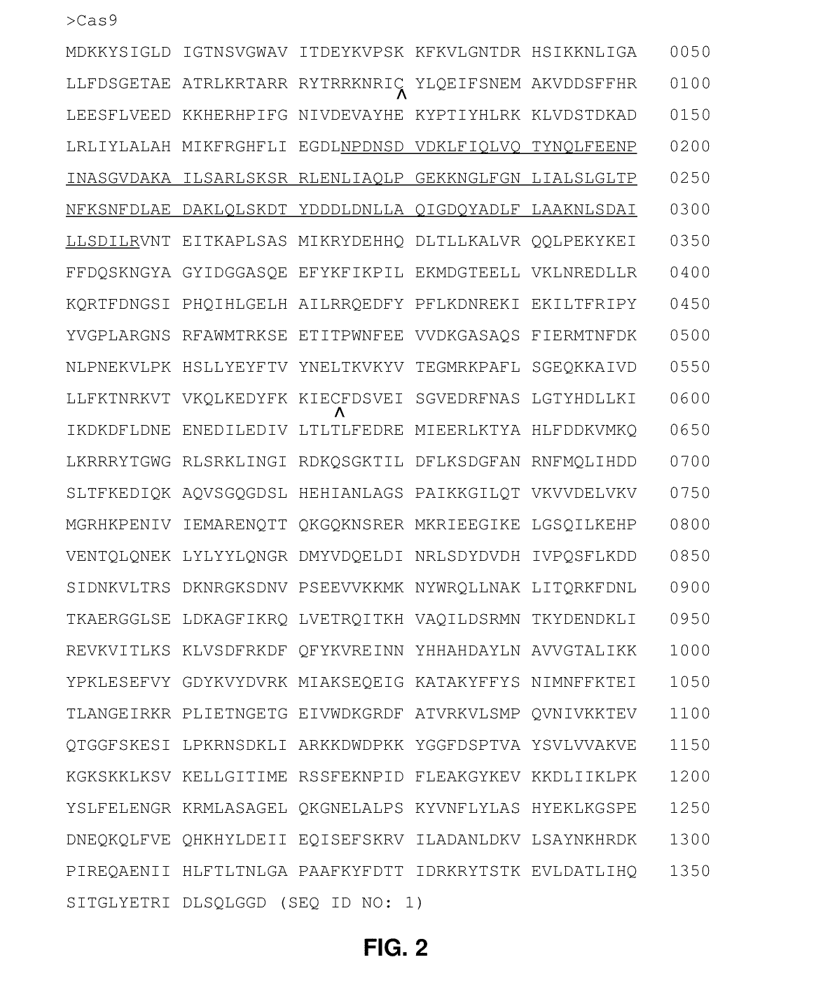

FIG. 2 depicts the amino acid sequence of Cas9 with some marks to identify certain portions of that protein that may be modified. In FIG. 2, carets call out two cysteines that are referenced later. Underlining indicates a subset of amino acids that may be most preferable for linkages, deletion, substitution, or other modification. It may be that the underlined portion represents a segment of the polypeptide that is not essential for the certain functions of Cas9 and that may be modified while preserving the utility of Cas9 in an antiviral therapeutic composition.

Functional groups that may be targeted for bioconjugation include primary amines (e.g., as presented by the N-terminus of a polypeptide or the side chain of a lysine reside), sulfhydryls (e.g., as presented by the side chain of a cysteine residue), and carboxylic acids (e.g., as presented by a glutamic acid or aspartic acid side chain). Primary amines exists at the N-terminus of each polypeptide and in the side chain of lysine. At physiological conditions, an amine will be positively charged and may be expected to be on the outer surface of a protein. Chemical groups that target primary amines include N-hydroxysuccinimide esters (NHS esters) and imidoesters (both commonly used) as well as cyonates, sulfonyl chlorides, aldehydes, carbodiimides, acyl azides, anhydrides, fluorbenzene, carbonates, epoxide, and fluoropheyl ester. An effective reaction uses methyl-PEGn-NHS ester to PEGylate primary amines with an NHS leaving group. That chemistry may be used to (relatively promiscuously) PEGylate N-terminal amines and Lysine side chains. Amino acid residues in Cas9 that may be linked to (e.g., PEGylated) include K3, K4, K26, K30, K31, K33, K44, K45, K65, K76, K92, K111, K112, K131, K140, K141, K148, K163, K183, K209, K218, K233, K234, K253, K263, K268, K294, K314, K323, K336, K346, K348, K374, K377, K382, K392, K401, K434, K439, K442, K468, K484, K500, K506, K510, K526, K528, K536, K545, K546, K554, K558, K562, K565, K570, K571, K599, K602, K604, K637, K649, K652, K665, K673, K677, K684, K705, K710, K734, K735, K742, K749, K755, K772, K775, K782, K789, K797, K810, K848, K855, K862, K866, K877, K878, K880, K890, K896, K902, K913, K918, K929, K942, K948, K954, K959, K961, K968, K974, K999, K1000, K1014, K1020, K1024, K1031, K1035, K1047, K1059, K1076, K1085, K1096, K1097, K1107, K1113, K1118, K1123, K1124, K1129, K1130, K1148, K1151, K1153, K1155, K1156, K1158, K1161, K1176, K1185, K1188, K1191, K1192, K1197, K1200, K1211, K1222, K1231, K1244, K1246, K1255, K1263, K1278, K1289, K1296, K1300, K1325, K1334, and K1340.

Carboxylic acids exist at the C-terminus of a polypeptide chain as well as in side chains of aspartic and glutamic acid. Carboxyls are usually on the surface of a protein. Carboiimides will react with this functional group. Residues that may be modified using, for example, a carbodiimide may include D10, D23, D39, D54, D94, D95, D110, D124, D144, D150, D173, D177, D180, D182, D207, D257, D261, D269, D271, D272, D273, D275, D284, D288, D298, D304, D326, D331, D353, D364, D384, D397, D406, D428, D435, D483, D499, D550, D567, D576, D585, D596, D603, D605, D608, D614, D618, D628, D644, D645, D672, D681, D686, D699, D700, D707, D718, D745, D821, D825, D829, D835, D837, D839, D849, D850, D853, D861, D868, D912, D936, D944, D947, D965, D969, D986, D1017, D1075, D1079, D1117, D1125, D1127, D1135, D1180, D1193, D1251, D1267, D1284, D1288, D1299, D1328, D1332, D1344, D1361, and D1368. Residues that may be modified in such fashion may also include E24, E57, E60, E84, E89, E102, E103, E208, E209, E114, E125, E130, E171, E197, E198, E223, E232, E260, E311, E327, E345, E349, E370, E371, E381, E387, E388, E396, E418, E427, E438, E441, E470, E471, E479, E480, E493, E505, E516, E523, E532, E543, E566, E573, E579, E584, E610, E611, E613, E617, E627, E630, E633, E634, E706, E722, E746, E757, E762, E766, E779, E785, E786, E790, E798, E802, E809, E827, E873, E874, E904, E910, E923, E945, E952, E1005, E1007, E1026, E1028, E1049, E1056, E1064, E1068, E1071, E1108, E1150, E1170, E1175, E1183, E1189, E1205, E1207, E1219, E1225, E1243, E1250, E1253, E1260, E1268, E1271, E1275, E1304, E1307, E1341, and E1357.

In preferred embodiments, the programmable nuclease 107 is an RNA-guided nuclease having a functional group modified by a secondary moiety 111.

The RNA-guided nuclease may be Cas9 or a modified Cas9 having an amino acid sequence at least 90% similar to Cas9. The nuclease may be codon-optimized for the host. The programmable nuclease may be present in ribonucleoprotein form with the nuclease complexed with a guide RNA. A portion of the guide RNA is complementary to a target in a viral genome and not substantially complementary to any part of a human genome. The secondary moiety may be attached to the side chain through a linker, which may include a disulfide bond; a thioether; an amine bond; a hydrazine linkage; an amide bond; an imidoester; a peptide bond; maleimide; polyethylene glycol (PEG); BM(PEG)n with 1<n<9; and biotin.

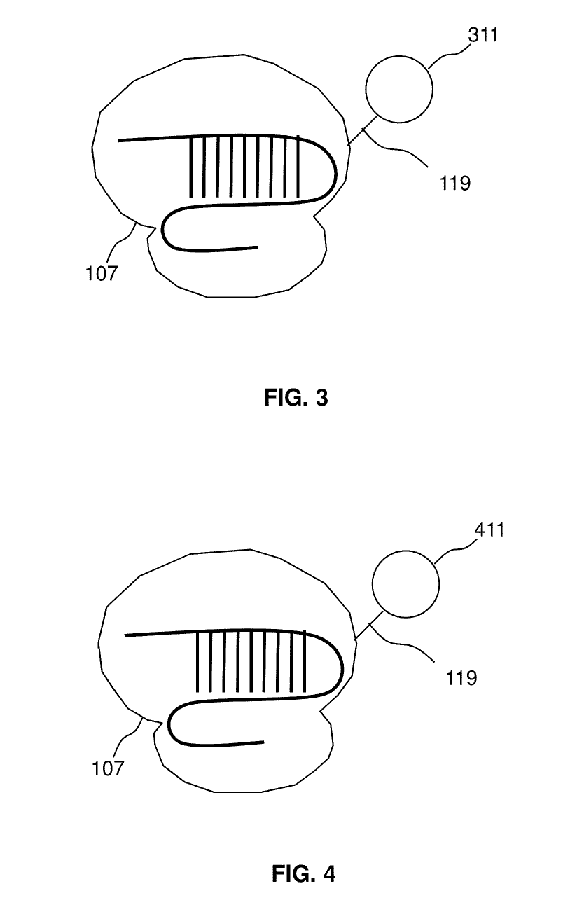

FIG. 3 diagrams a programmable nuclease 107 linked to an aptamer 311 via a chemical linkage, which may include a linker 119. Aptamers exhibit many desirable properties for targeted drug delivery, such as ease of selection and synthesis, high binding affinity and specificity, low immunogenicity, and versatile synthetic accessibility. An aptamer may be linked to the programmable nuclease 107 to bring the nuclease to a target or keep it there. Additionally or alternatively, linkage to an aptamer may decrease immunogenicity of the composition relative to a naked nuclease.

FIG. 4 diagrams a programmable nuclease linked to at least a portion of an apolipoprotein E (apoE) protein 411, optionally through an intervening linker 119. Studies suggest an APOE-derived peptide could help deliver therapeutics across the blood-brain barrier (BBB). The BBB is a barrier for many high-molecular-weight compounds. Protein concentration in the cerebrospinal fluid (CSF) is equal to 0.4 mg/mL, which is significantly lower than that in the blood plasma (70 mg/mL). Particles with covalently bound apoE on their surface have exhibited ability to transport through the BBB. For more, see Georgieva et al., 2014, Smuggling drugs into the brain: an overview of ligands targeting transcytosis for drug delivery across the blood-brain barrier, Pharmacuetics 6(4):557-583 and Zensi et al., 2009, Albumin nanoparticles targeted with Apo E enter the CNS by transcytosis and are delivered to neurons, J Control Release 137:78-86, both incorporated by reference.

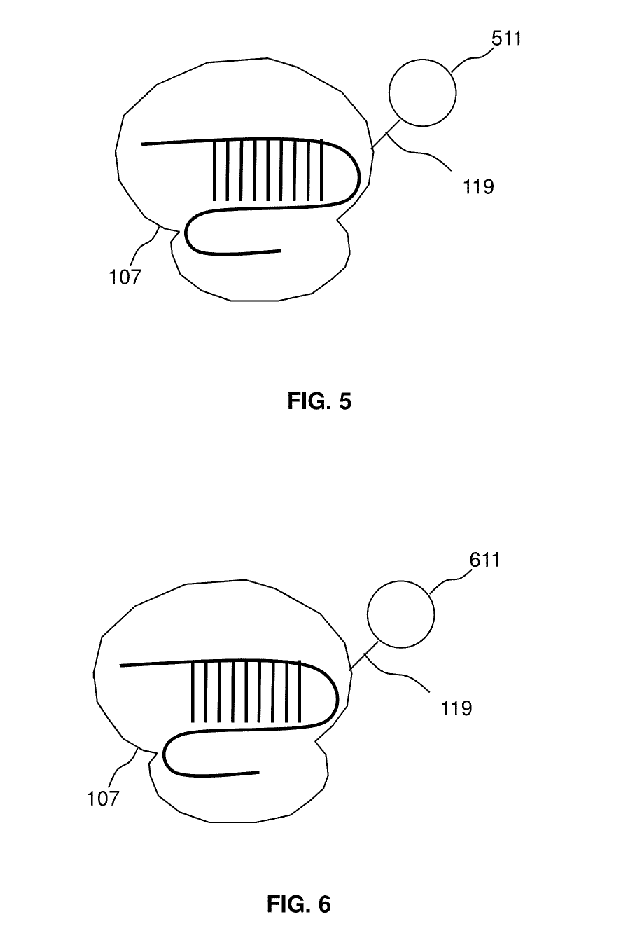

FIG. 5 diagrams a programmable nuclease linked to at least a portion of an Fc region 511 of an immunoglobulin, optionally through a linker 119. The presence of an Fc domain confers a marked increase on plasma half-life, prolonging therapeutic effect. In one approach, the programmable nuclease is fused to the amino terminus of Fc.

FIG. 6 diagrams a programmable nuclease linked to albumin 611. Albumin may increase serum half-life and may decrease immunogenicity. Albumin is taken up by cells through pinocytosis and is protected from degradation through pH-dependent binding to FcRn in endosomes. The interaction with the FcRn allows albumin to then be recycled back to the cell surface where it is released into circulation. The fusion of a programmable nuclease to albumin may extend half-life significantly, resulting in improved pharmacokinetic properties.

### ii. RECOMBINANT FUSION PROTEINS

Embodiments of the invention provide a programmable nuclease linked to a secondary moiety within a recombinant fusion protein as a composition for treating a viral infection. Such a composition may be preferred for its amenability to known approaches to synthesis. A gene for the secondary moiety and a gene for the programmable nuclease may be combined via known genetic engineering techniques. The resulting recombinant gene may be expressed using, for example, E. coli or another suitable medium. Optionally, a linker segment may be included in the recombinant gene to provide a proteinaceous linker between the programmable nuclease and the secondary moiety. The gene is expressed and collected (e.g., purified, isolated, and optionally provided within a pharmaceutical carrier or solution). Where the programmable nuclease is an RNA-guided nuclease, a gRNA is provided to complex with the nuclease.

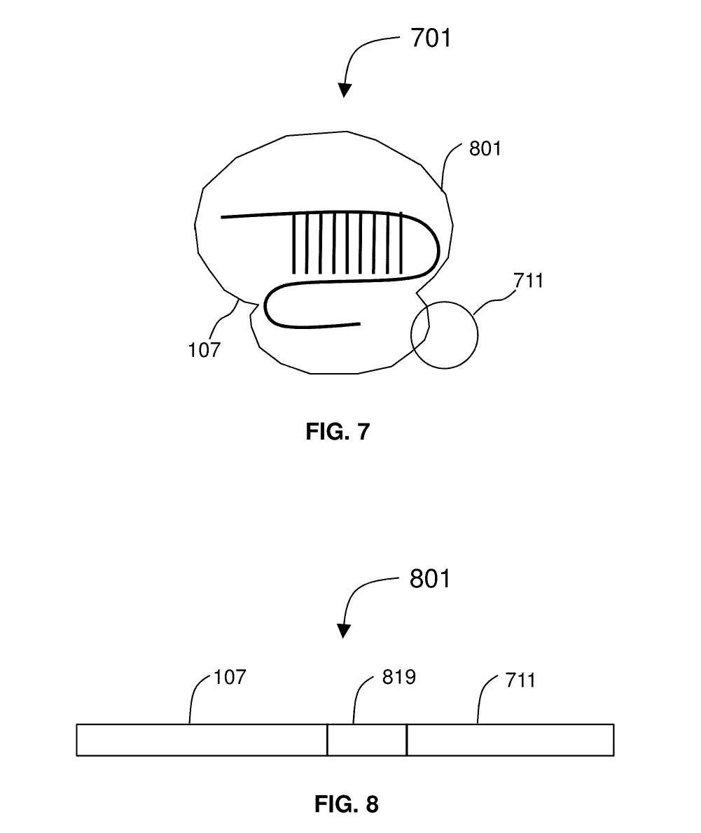

FIG. 7 shows a composition 701 for treating a viral infection that includes a programmable nuclease 107 linked to a secondary moiety 711 within a recombinant protein 801. In the illustrated embodiment, the RNA-guided nuclease is Cas9 and will be complexed with a guide RNA 115 to form a ribonucleoprotein when the composition 701 is used. A portion of the guide RNA 115 is complementary to a target in a viral genome and not substantially complementary to any part of a human genome.

FIG. 8 diagrams a primary structure of a recombinant protein 801 to be used in composition 701. The recombinant protein 801 includes the programmable nuclease 701 and the secondary moiety 711. The recombinant protein 801 optionally includes a linker 819 between the secondary moiety 711 and the RNA-guided nuclease. Other nucleases may be used including, for example, ZFNs, TALENs, Cpf1, or a modified Cas9 having an amino acid sequence at least 90% similar to Cas9. Any suitable linker may be included. For example, the linker may include a plurality of glycine residues. In the illustrated embodiment, the secondary moiety includes at least a portion of an apolipoprotein E (apoE) protein

Where the programmable nuclease is an RNA-guided nuclease (e.g., Cas9 or a modified Cas9 having an amino acid sequence at least 90% similar to Cas9), the RNA-guided nuclease portion of the recombinant protein may be present in ribonucleoprotein form with the nuclease complexed with a guide RNA (as shown in FIG. 7), in which a portion of the guide RNA 115 is complementary to a target in a viral genome and not substantially complementary to any part of a human genome. An exemplary flexible linker 819 may include a plurality of glycine residues.

Any of the recombinant fusion proteins described herein may be provided through the use of a nucleic acid vector such as a plasmid. Additionally, some aspects of the invention provide an antiviral therapy that includes a nucleic acid vector encoding a modified programmable nuclease for delivery to viral-infected cells. In preferred embodiments, the nucleic acid vector is a plasmid and the encoded nuclease is an RNA-guided nuclease such as Cas9, a modified Cas9 (at least 90% similar to wt Cas9), Cpf1, or a modified Cpf1. The programmable nuclease may be encoded as part of a recombinant gene on the plasmid. The recombinant gene includes a portion coding for the programmable nuclease and a portion coding for a secondary moiety, optionally connected to each other through a linker.

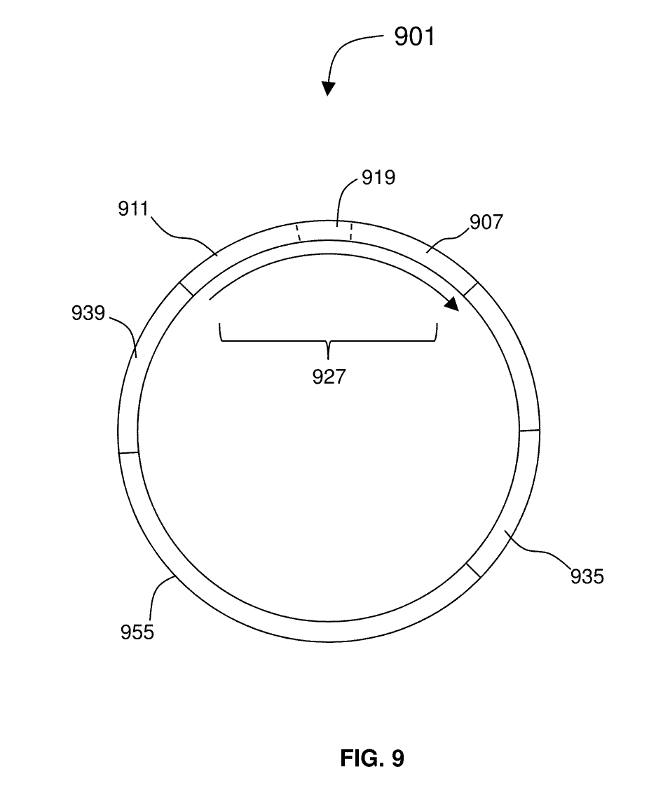

FIG. 9 diagrams a composition 901 for treating a viral infection that includes a nucleic acid vector 901 encoding a modified programmable nuclease for delivery to viral-infected cells. In the depicted embodiment, the nucleic acid vector 901 is a plasmid that includes a recombinant gene 927 preferably under control of a promoter 939. The plasmid may also include a viral origin of replication 935 to support maintenance of the plasmid preferentially in viral-infected cells. The recombinant gene 927 includes a programmable nuclease segment 907 and a secondary moiety segment 911 separated by a linker 919 (the linker is optional). The programmable nuclease segment 907 may preferably code for an RNA-guided nuclease such as Cas9, a modified Cas9 (e.g., at least 90% similar to an unmodified Cas9), Cpf1, or a modified Cpf1 (e.g., at least 90% similar to an unmodified Cpf1). It will be appreciated that the recombinant protein has portions other than the programmable nuclease and thus that, in the context of a recombinant fusion protein, any reference to a modified programmable nuclease and a percent similarity refers only to that portion of the recombinant fusion protein that is homologous to the unmodified programmable nuclease. Where the programmable nuclease segment 907 codes for an RNA-guided nuclease, the plasmid may also include one or a plurality of a guide RNA segment 955, which includes portions that correspond to targets in genetic material of a virus. When the guide RNA segment 955 is transcribed, the product is a gRNA with a portion substantially complementary to a target in viral genetic material, preferably with no match in a human genome.

In certain embodiments, vector 901 is a plasmid, programmable nuclease segment 907 codes for Cas9 and is at least a 98% match to SEQ ID NO: 1; secondary moiety segment 911 codes for an Fc portion of an immunoglobulin; linker 919 include (GGGS)3; guide RNA segment 955 includes a 20 nucleotide segment that is at least a 70% match to a segment in a genome of a virus adjacent to a protospacer adjacent motif (PAM) (e.g., NGG); and the viral origin of replication 935 is an origin of replication from the genome of a virus. The virus may be selected from Human papillomavirus (HPV), Hepatitis B virsus, Cytomegalovirus, herpes simplex virus, Epstein Barr virus, for example. These certain embodiments may be preferred where the nucleic acid vector 901 is part of an antiviral therapeutic composition to be delivered to infected cells.

In some embodiments, vector 901 is a plasmid that includes recombinant gene 927, which itself includes a programmable nuclease segment 907 and a secondary moiety segment 911, optionally separated by a linker 919. The programmable nuclease segment 907 may preferably code for an RNA-guided nuclease such as Cas9, a modified Cas9 (at least 90% similar to wt Cas9), Cpf1, or a modified Cpf1. In these some embodiments, the guide RNA segment 955 and the viral origin of replication 935 are preferably omitted. Any suitable promoter 939 (e.g., U6 promoter) may be included. These some embodiments may be preferred where the recombinant gene 927 is to be expressed e.g., in culture (for example, in E. coli, yeast, or a Lactobacillus) to produce a recombinant protein for use in an antiviral therapeutic composition. Where the programmable nuclease segment 907 codes for an RNA-guided nuclease, the expressed recombinant protein is preferably complexed with a gRNA to form into an active ribonucleoprotein (RNP) that includes the secondary moiety, such as an Fc chain of an immunoglobulin, or any other polypeptide such as those described herein. (Such as, for example, albumin or biotin.)

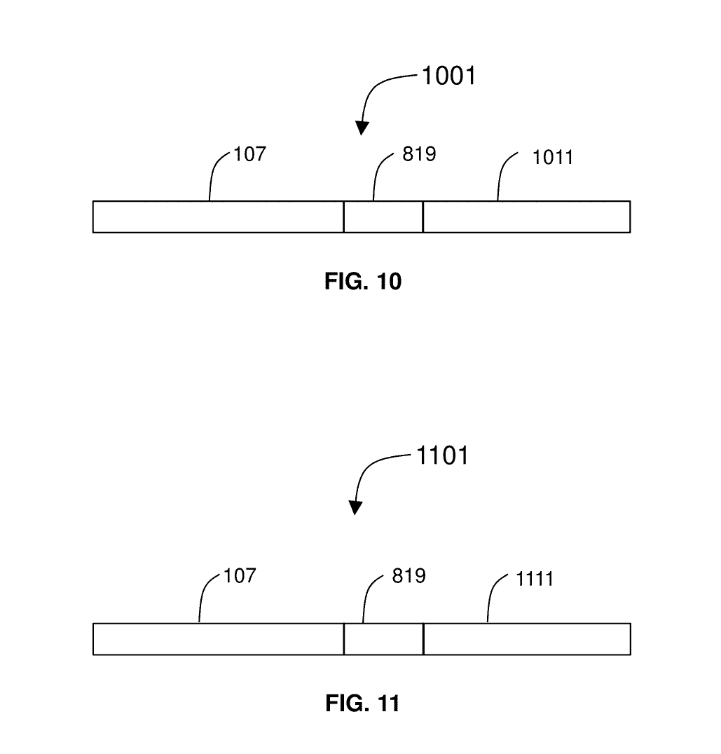

FIG. 10 diagrams a composition 1001 for treating a viral infection that includes a programmable nuclease 107 linked to at least a portion of albumin 1011 in a recombinant protein. A linker 819 is optionally included.

FIG. 11 diagrams a composition 1101 for treating a viral infection that includes a programmable nuclease 107 linked to biotin 111 in a recombinant protein. A linker 819 is optionally included.

### iii. LINKERS

In embodiments of the invention, a secondary moiety is linked to a programmable nuclease through a linker. A linker may be chosen for its properties. For example, for a polypeptide linker (e.g., within a recombinant fusion protein) to be flexible it may be provided with a plurality of glycine resides (e.g., >30% or >50%). For a more rigid polypeptide linker, it may be desirable to include a plurality of proline residues. The linker may be biodegradable.

In some embodiments, the linker is cleavable. For example, the linker may include an enzyme cleavage region. Where a polypeptide linker is used, an enzyme cleave region can be the target of a protease.

In certain embodiments, the secondary moiety is non-covalently bound to the programmable nuclease. For example, either the programmable nuclease or the secondary moiety may be biotinylated and the secondary moiety may thus be non-covalently bound to the programmable nuclease through a biotin/streptavidin linkage.

a. Protein Linkers

A composition for treating a viral infection may include a programmable nuclease covalently linked to a secondary moiety through a protein linker. Some recombinant fusion proteins are composed of two or more functional domains joined by linker peptides. The linker serves to connect the protein moieties, and also provide many other functions, such as maintaining cooperative inter-domain interactions or preserving biological activity. The natural length of linkers in multi-domain proteins is about 6 to 10 residues on average. Preferred residues for linkers include threonine (Thr), serine (Ser), proline (Pro), glycine (Gly), aspartic acid (Asp), lysine (Lys), glutamine (Gln), asparagine (Asn), and alanine (Ala), arginine (Arg), phenylalanine (Phe), and glutamic acid. I.e., preferably residues are polar (charged or uncharged).

Proline may be included to give the linker rigidity. It is though that the lack of an amide hydrogen, as well as the cyclic side chain, limit proline's ability to participate in promiscuous hydrogen bonding and restrict its flexibility. The small, polar amino acids, such as Thr, Ser, and Gly are thought to be favorable for providing good flexibility due to their small sizes, and also help maintain stability of the linker structure in the aqueous solvent through formation of hydrogen bonds with water. For flexibility, the linker may include a plurality of glycine residues. In some embodiments, the linker comprises a plurality of threonine and serine residues.

An exemplary flexible linker may include (GGGS)3.

An exemplary rigid linker may include (EAAAK)n (n=1-3).

Thus in some embodiments, compositions of the invention include—or include nucleic acids that encode: GGGS (SEQ ID NO: 2); or EAAAK (SEQ ID NO: 3).

b. Non-Protein Chemical Linkers

A composition for treating a viral infection may include a programmable nuclease linked to a secondary moiety through a non-protein chemical linker.

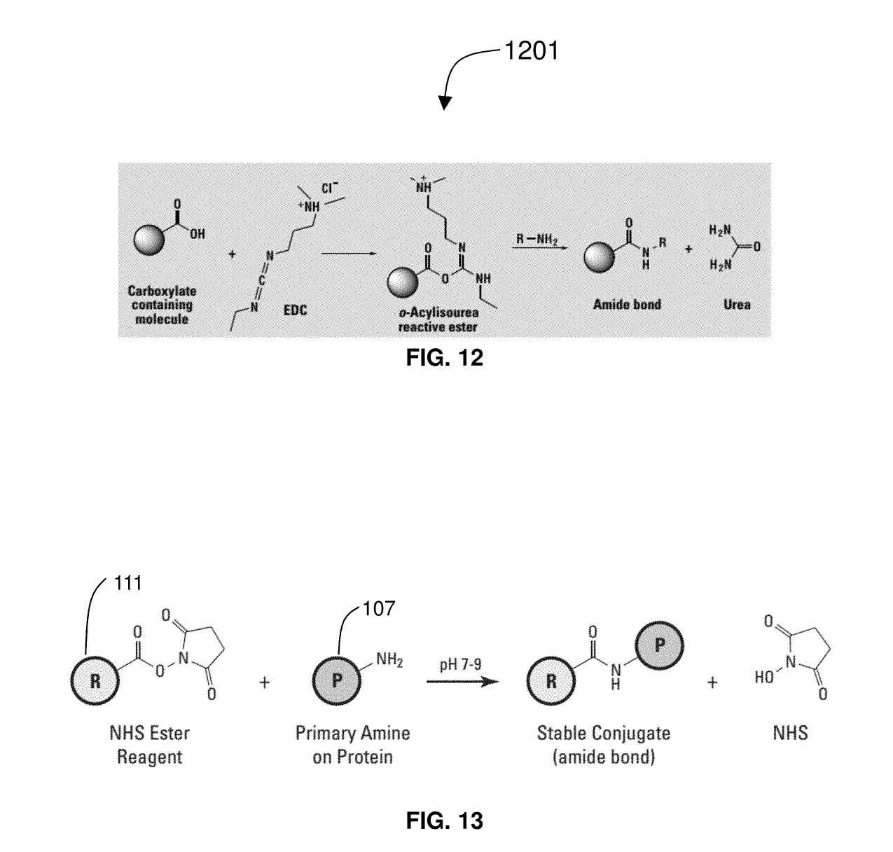

FIG. 12 diagrams a chemical pathway 1201 for attaching a secondary moiety at a carbonyl. The depicted chemistry may be used to attach a secondary moiety 111 to a carboxylic acid side chain of an amino acid in the programmable nuclease 107. In pathway 1201, the target amino acid is exposed to 1-ethyl-3-(3-dimethylaminopropyl)carbodiimide (EDC) to form an ester. The secondary moiety 111 with an available primary amine is introduced and forms an amide bond with the carboxylic acid side chain.

Where, for example, the programmable nuclease is Cas9, pathway 1201 may be used to attach the secondary moiety 111 to one or more of the following residues: D10, D23, D39, D54, D94, D95, D110, D124, D144, D150, D173, D177, D180, D182, D207, D257, D261, D269, D271, D272, D273, D275, D284, D288, D298, D304, D326, D331, D353, D364, D384, D397, D406, D428, D435, D483, D499, D550, D567, D576, D585, D596, D603, D605, D608, D614, D618, D628, D644, D645, D672, D681, D686, D699, D700, D707, D718, D745, D821, D825, D829, D835, D837, D839, D849, D850, D853, D861, D868, D912, D936, D944, D947, D965, D969, D986, D1017, D1075, D1079, D1117, D1125, D1127, D1135, D1180, D1193, D1251, D1267, D1284, D1288, D1299, D1328, D1332, D1344, D1361, D1368, E24, E57, E60, E84, E89, E102, E103, E208, E209, E114, E125, E130, E171, E197, E198, E223, E232, E260, E311, E327, E345, E349, E370, E371, E381, E387, E388, E396, E418, E427, E438, E441, E470, E471, E479, E480, E493, E505, E516, E523, E532, E543, E566, E573, E579, E584, E610, E611, E613, E617, E627, E630, E633, E634, E706, E722, E746, E757, E762, E766, E779, E785, E786, E790, E798, E802, E809, E827, E873, E874, E904, E910, E923, E945, E952, E1005, E1007, E1026, E1028, E1049, E1056, E1064, E1068, E1071, E1108, E1150, E1170, E1175, E1183, E1189, E1205, E1207, E1219, E1225, E1243, E1250, E1253, E1260, E1268, E1271, E1275, E1304, E1307, E1341, and E1357. Where the programmable nuclease is a modified Cas9, homologous residues may be targeted.

FIG. 13 diagrams a chemical pathway 3101 for attaching a secondary moiety 111 to a programmable nuclease 107 at a primary amine. This is a hydrazide reaction. Note that for any of the depicted pathways, the identity of the secondary moiety 111 and the programmable nuclease 107 may be reversed.

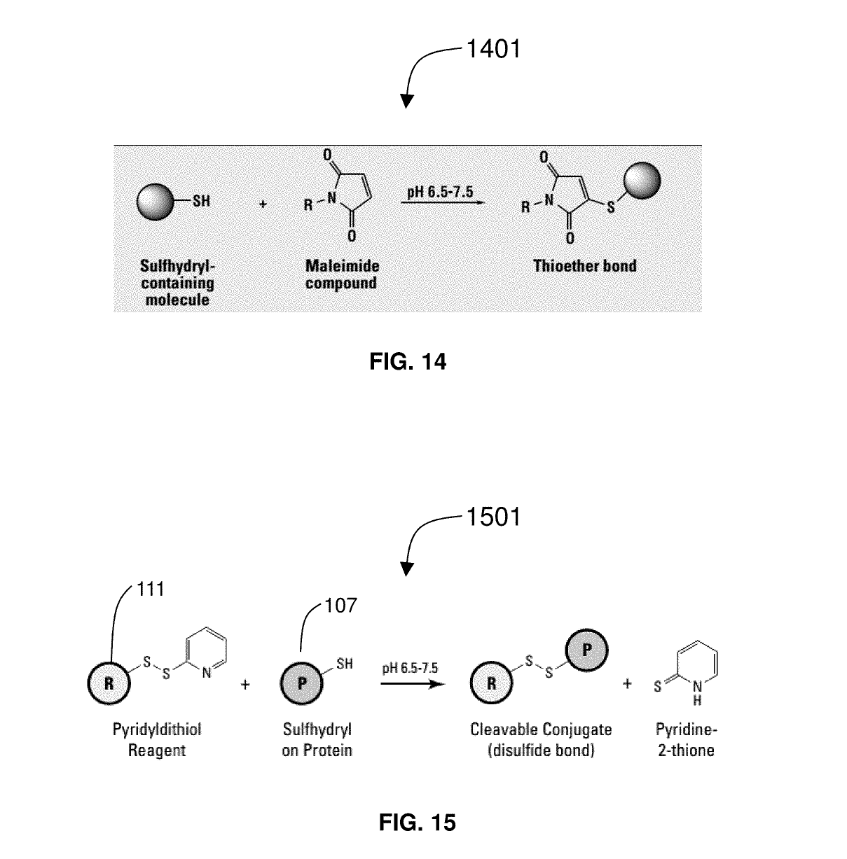

FIG. 14 shows a pathway 1401 for a maleimide reaction to attach a secondary linker via a thioether bond.

FIG. 15 shows a pathway 1501 for linking a secondary moiety via a disulfide bond.

Pathways 1401 and 1501 may be used for connecting a secondary moiety to a cysteine residue in the programmable nuclease (e.g., C80 or C574 in Cas9 or homologs thereof).

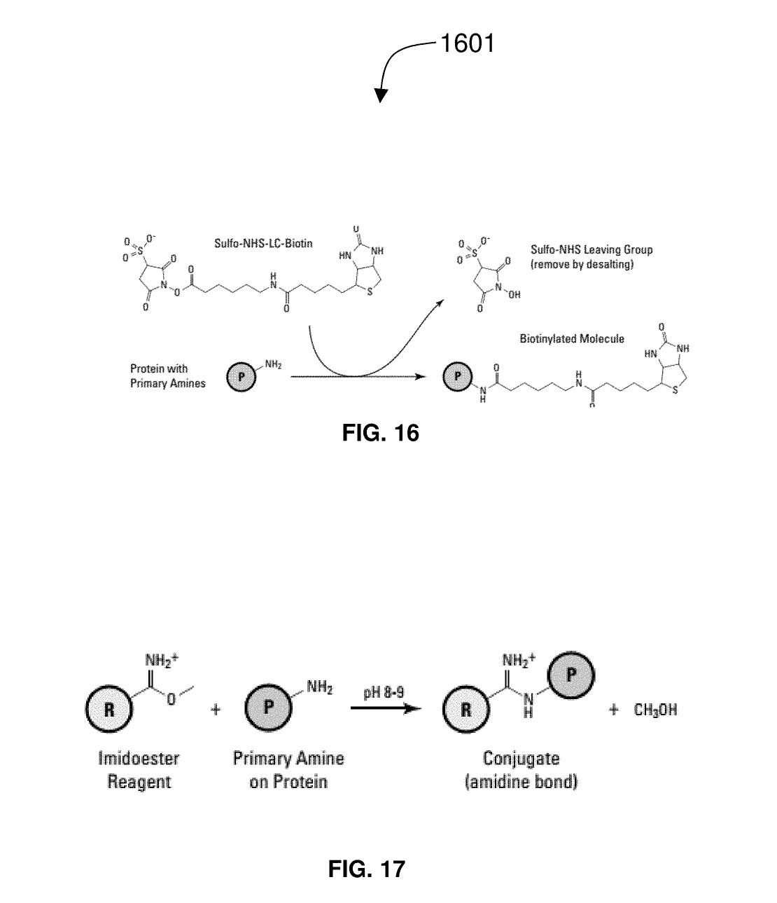

FIG. 16 shows a pathway 1601 for biotinylating a primary amine. Pathway 1601 may be used to attach biotin as a secondary moiety 111 to a programmable nuclease 107 at any accessible primary amine. For example, where the programmable nuclease 107 is Cas9, Cas9 may be biotinylated via pathway 1601 at the N-terminus, K3, K4, K26, K30, K31, K33, K44, K45, K65, K76, K92, K111, K112, K131, K140, K141, K148, K163, K183, K209, K218, K233, K234, K253, K263, K268, K294, K314, K323, K336, K346, K348, K374, K377, K382, K392, K401, K434, K439, K442, K468, K484, K500, K506, K510, K526, K528, K536, K545, K546, K554, K558, K562, K565, K570, K571, K599, K602, K604, K637, K649, K652, K665, K673, K677, K684, K705, K710, K734, K735, K742, K749, K755, K772, K775, K782, K789, K797, K810, K848, K855, K862, K866, K877, K878, K880, K890, K896, K902, K913, K918, K929, K942, K948, K954, K959, K961, K968, K974, K999, K1000, K1014, K1020, K1024, K1031, K1035, K1047, K1059, K1076, K1085, K1096, K1097, K1107, K1113, K1118, K1123, K1124, K1129, K1130, K1148, K1151, K1153, K1155, K1156, K1158, K1161, K1176, K1185, K1188, K1191, K1192, K1197, K1200, K1211, K1222, K1231, K1244, K1246, K1255, K1263, K1278, K1289, K1296, K1300, K1325, K1334, K1340, or some combination thereof. Where the programmable nuclease 107 is a modified Cas9 or is homologous to Cas9, it may be biotinylated at positions homologous to those just listed.

FIG. 17 illustrates the reaction of an imidoester crosslinker with a primary amine to form an amidine bond.

A linker 119 may provide functionality such as flexibility, rigidity (e.g., even a mixture of both flexibility and rigidity at different points along it), solubility, cleavage targets, binding targets, others, or combinations thereof. In some embodiments, the linker 119 is included to provide a spacer arm. The spacer arm is the chemical chain between two groups. The length of a spacer arm (e.g., in angstroms) determines how flexible a conjugate will be. Longer spacer arms have greater flexibility, reduced steric hindrance, and offer more sites for potential nonspecific binding. Spacer arms can range from zero length to >100 angstroms. The molecular composition of a crosslinkers spacer arm can affect solubility and nonspecific binding. Some linkers have spacer arms that contain hydrocarbon chains or polyethylene glycol (PEG) chains. Hydrocarbon chains are not water soluble and typically require an organic solvent such as DMSO or DMF for suspension. Those crosslinkers are suited for penetrating the cell membrane and performing intercellular crosslinking because they are hydrophobic and uncharged. If a charged sulfonate group is added to the termini of such crosslinkers, a water soluble analogue is formed.

Certain exemplary categories of cross-linkers use bismaleimide-activated PEG (BM(PEG)n) or bis(succinimidyl) PEG (BS(PEG)n). Canonically, BM(PEG)n cross-links sulfhydryls and BS(PEG)n cross-links amines, although variations will be understood by one of skill in the art.

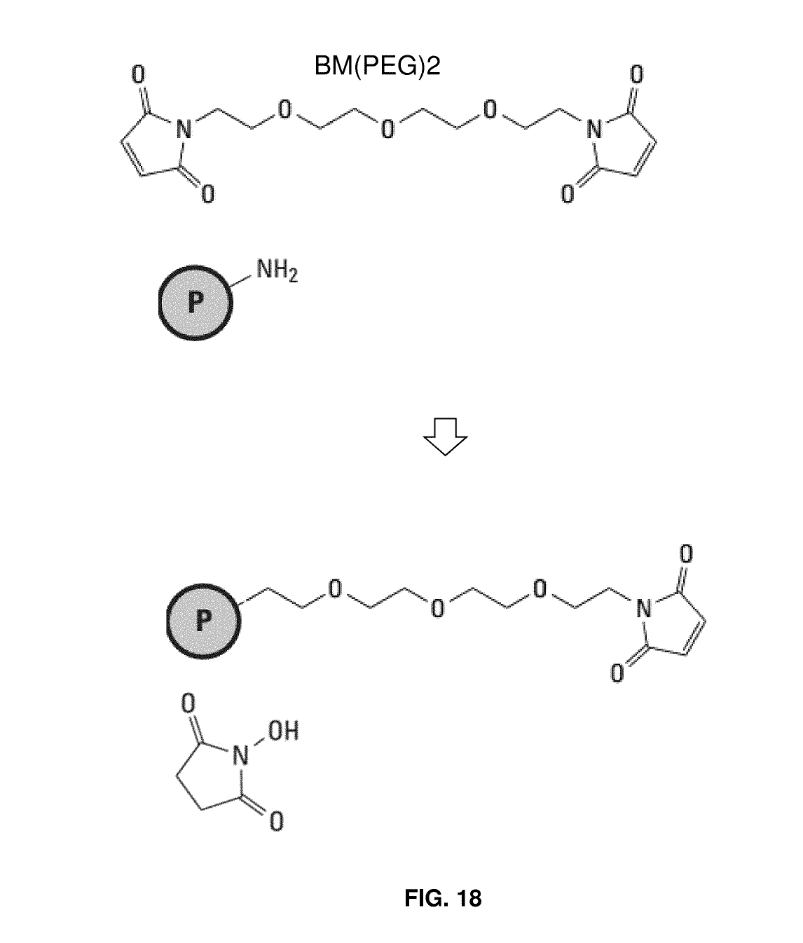

FIG. 18 shows the use of BS(PEG)2 to link to an amine of a programmable nuclease. The unlinked succinimidyl portion at the bottom of FIG. 18 may further be reacted to a primary amine in a secondary moiety 111. It may be preferable for most technological and therapeutic applications to use BS(PEG)n or BM(PEG)n with 1<n<9, although related PEG-based chemistries will be understood by one of skill in the art and are included in the invention.

Using any of the foregoing pathways or other chemical pathways, a secondary moiety may be attached to a programmable nuclease, optionally through a linker, at an amino acid with a side chain comprising an amine, a carboxyl, a sulfhydryl, or a carbonyl. For example, the secondary moiety or the linker may be attached to the programmable nuclease at an amino acid in the nuclease such as lysine, cysteine, aspartic acid, or glutamic acid.

In some embodiments, a programmable nuclease is linked to a secondary moiety through a click reaction product such as one more five-membered rings or acyclic derivatives thereof. Click chemistry includes a class of biocompatible reactions intended primarily to join substrates of choice with specific biomolecules. Click chemistry provides methods joining small modular units. In general, click reactions usually join a biomolecule and a secondary moiety. Typical click reactions occur in one pot, are not disturbed by water, make unremarkable byproducts, and are driving quickly and irreversibly to high yield of a single click reaction product, with high reaction specificity (in some cases, with both regio- and stereo-specificity). Click reaction products are physiologically stable with only non-toxic byproducts. In one example, the Azide-Alkyne Huisgen Cycloaddition is a 1,3-dipolar cycloaddition between an azide and a terminal or internal alkyne to give a 1,2,3-triazole. The 1,3-dipolar cycloaddition is a chemical reaction between a 1,3-dipole and a dipolarophile to form a five-membered ring. Linking a programmable nuclease to a secondary moiety via click chemistry can create a linker that includes, as the click reaction product, one or more five-membered rings or acyclic derivatives thereof. This 1,3-dipolar cycloaddition is an important route to the regio- and stereoselective synthesis of five-membered heterocycles and their ring-opened acyclic derivatives.

The 1,3-dipolar cycloaddition between organic azides and terminal alkynes, e.g., for bioconjugation, may proceed by a copper(I)-catalyzed version of the Huisgen reaction, CuAAC (for Copper-catalyzed Azide-Alkyne Cycloaddition), which proceeds readily in mild conditions that can approximate physiological conditions. Click chemistry may be bioorthogonal: azides and alkynes are typically not found in biomolecules discussed herein and can be selectively reacted. For discussion see Hein et al., 2009, Click chemistry, a powerful tool for pharmaceutical sciences, Pharm Res 25(10):2216-2230 and McCombs & Owen, 2015, Antibody drug conjugates: design and selection of linker, payload and conjugation chemistry, AAPS J 17(2):339-51, both incorporated by reference.

### iv. SUBSTITUTIONS

In some embodiments, a programmable nuclease includes one or more mutations to improve utility in an antiviral therapeutic. For example, where the protein is Cas9 or a modified Cas9, it may be beneficial to delete any or all residues from N175 to R307 (inclusive). The residues from N175 to R307 in Cas9 are underlined in FIG. 2. It may be found that a smaller, or lower-molecular mass, version of the nuclease is more effective. In some embodiments, the nuclease comprises at least one substitution relative to a naturally-occurring version of the nuclease. For example, where the protein is Cas9 or a modified Cas9, it may be beneficial to mutate C80 or C574 (or homologs thereof, in modified proteins with indels). In Cas9, desirable substitutions may include any of C80L, C80I, C80V, C80A, C80K, C574E, C574D, C574N, C574Q (in any combination) and in particular C80L and/or C574E. Substitutions may be included to reduce intracellular protein binding of the nuclease. Additionally or alternatively, substitutions may be included to reduce off-target toxicity of the composition.

v. Methods

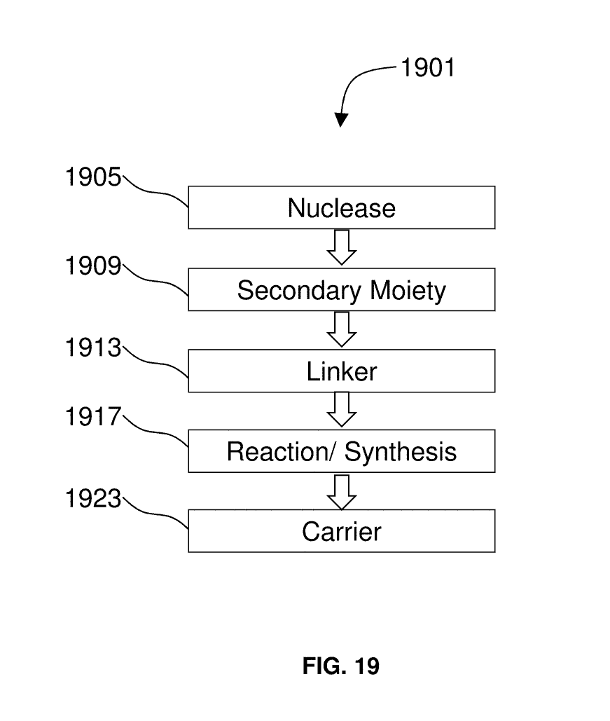

FIG. 19 charts steps of a method 1901 of preparing an antiviral compositions. The method 1901 includes the steps of providing 1905 a programmable nuclease and providing 1909 a secondary moiety. Optionally, the method 1901 includes the step of providing 1913 a linker. The method 1901 includes the steps of providing the programmable nuclease 107 linked to the secondary moiety 111, optionally through the linker. In some embodiments, the composition includes a recombinant fusion protein that includes the programmable nuclease 107 and the secondary moiety 111. In such cases, the method 1901 includes providing the linked programmable nuclease 107 and the secondary moiety 111 by synthesizing the recombinant fusion protein from a recombinant gene. In certain embodiments, the composition includes the programmable nuclease 107 linked to the secondary moiety 111 via a chemical linker. In such cases, the method 1901 includes providing the linked programmable nuclease 107 and the secondary moiety 111 by performing the appropriate chemical reaction including any of those described elsewhere herein. Optionally, the method 1901 includes providing the linked programmable nuclease 107 and secondary moiety 111 with a suitable carrier such as a liposome, a solution, cream, ointment, or other pharmaceutically or therapeutically acceptable carrier.

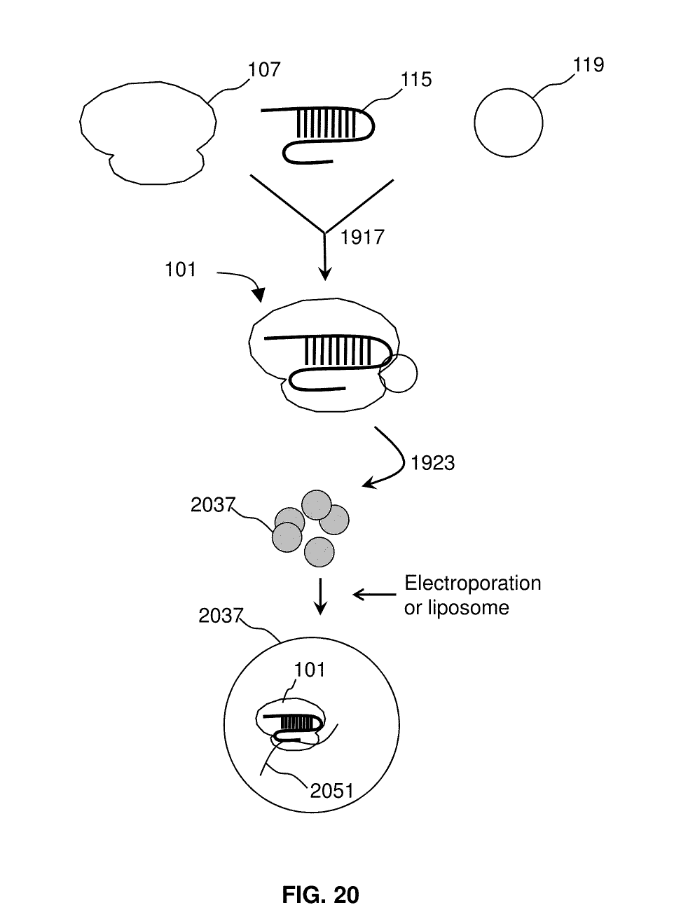

FIG. 20 shows preparation of an antiviral composition according to some embodiments of method 1901. The programmable nuclease and secondary moiety 111 are obtained. Those elements are linked 1917 to form the composition 101. Where the nuclease is an RNA-guided nuclease, a gRNA 115 is provided.

In preferred embodiments, the composition 101 includes the programmable nuclease 107 in the form of an active ribonucleoprotein (RNP) linked to the secondary moiety (e.g., an active, PEGylated Cas9 RNP). The composition may be provided 1923 with a suitable carrier such as a liposome 2037. Thus in certain embodiments, the invention provides an active Cas9 RNP modified with a secondary moiety such as PEG, albumin, Fc, or ApoE and contained within a liposome. It may be found that a liposome-enclosed, active RNP form of a modified nuclease of Cas9 or a homolog thereof is preferable for delivery as an antiviral therapeutic. The modification may most preferably include that the RNP is linked to one or more of PEG, an aptamer, apoE, albumin, elastin, an Fc chain, biotin, lectin, a carbohydrate, a cell-penetrating peptide, an enzyme, a zinc finger, or others.

Compositions of the invention may be delivered by any suitable method include subcutaneously, transdermal, by hydrodynamic gene delivery, topically, or any other suitable method. In some embodiments, the composition is provided a carrier and is suitable for topical application to the human skin. The composition may be introduced into the cell in situ by delivery to tissue in a host. Introducing the composition into the host cell may include delivering the composition non-systemically to a local reservoir of the viral infection in the host, for example, topically.

Compositions of the invention may be delivered to an affected area of the skin in a acceptable topical carrier such as any acceptable formulation that can be applied to the skin surface for topical, dermal, intradermal, or transdermal delivery of a medicament. The combination of an acceptable topical carrier and the compositions described herein is termed a topical formulation of the invention. Topical formulations of the invention are prepared by mixing the composition with a topical carrier according to well-known methods in the art, for example, methods provided by standard reference texts such as, REMINGTON: THE SCIENCE AND PRACTICE OF PHARMACY 1577-1591, 1672-1673, 866-885 (Alfonso R. Gennaro ed.); Ghosh, T. K.; et al. TRANSDERMAL AND TOPICAL DRUG DELIVERY SYSTEMS (1997).

The topical carriers useful for topical delivery of the compound described herein can be any carrier known in the art for topically administering pharmaceuticals, for example, but not limited to, acceptable solvents, such as a polyalcohol or water; emulsions (either oil-in-water or water-in-oil emulsions), such as creams or lotions; micro emulsions; gels; ointments; liposomes; powders; and aqueous solutions or suspensions, such as standard ophthalmic preparations.

In certain embodiments, the topical carrier used to deliver the compositions described herein is an emulsion, gel, or ointment. Emulsions, such as creams and lotions are suitable topical formulations for use in accordance with the invention. An emulsion has at least two immiscible phases, one phase dispersed in the other as droplets ranging in diameter from 0.1 μm to 100 μm. An emulsifying agent is typically included to improve stability.

In another embodiment, the topical carrier is a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Polymer thickeners (gelling agents) that may be used include those known to one skilled in the art, such as hydrophilic and hydro-alcoholic gelling agents frequently used in the cosmetic and pharmaceutical industries. Preferably the gelling agent comprises between about 0.2% to about 4% by weight of the composition. The agent may be cross-linked acrylic acid polymers that are given the name carbomer. These polymers dissolve in water and form a clear or slightly hazy gel upon neutralization with a caustic material such as sodium hydroxide, potassium hydroxide, or other amine bases.

In another preferred embodiment, the topical carrier is an ointment. Ointments are oleaginous semisolids that contain little if any water. Preferably, the ointment is hydrocarbon based, such as a wax, petrolatum, or gelled mineral oil.

In another embodiment, the topical carrier used in the topical formulations of the invention is an aqueous solution or suspension, preferably, an aqueous solution. Well-known ophthalmic solutions and suspensions are suitable topical carriers for use in the invention. The pH of the aqueous topical formulations of the invention are preferably within the range of from about 6 to about 8. To stabilize the pH, preferably, an effective amount of a buffer is included. In one embodiment, the buffering agent is present in the aqueous topical formulation in an amount of from about 0.05 to about 1 weight percent of the formulation. Tonicity-adjusting agents can be included in the aqueous topical formulations of the invention.

The topical formulations of the invention can include acceptable excipients such as protectives, adsorbents, demulcents, emollients, preservatives, antioxidants, moisturizers, buffering agents, solubilizing agents, skin-penetration agents, and surfactants. Suitable protectives and adsorbents include, but are not limited to, dusting powders, zinc sterate, collodion, dimethicone, silicones, zinc carbonate, aloe vera gel and other aloe products, vitamin E oil, allatoin, glycerin, petrolatum, and zinc oxide. Suitable demulcents include, but are not limited to, benzoin, hydroxypropyl cellulose, hydroxypropyl methylcellulose, and polyvinyl alcohol. Suitable emollients include, but are not limited to, animal and vegetable fats and oils, myristyl alcohol, alum, and aluminum acetate. Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin. Chlorine dioxide (ClO2), preferably, stabilized chlorine dioxide, is a preferred preservative for use with topical formulations of the invention. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use in the invention include, but are not limited to, acetate buffers, citrate buffers, phosphate buffers, lactic acid buffers, and borate buffers. Suitable solubilizing agents include, but are not limited to, quaternary ammonium chlorides, cyclodextrins, benzyl benzoate, lecithin, and polysorbates. Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone.

In certain embodiments, compounds of the invention are conjugated to or encapsulated within nano-systems such as liposomes, albumin-based particles, PEGylated proteins, biodegradable polymer-drug composites, polymeric micelles, dendrimers, among others. See Davis et al., 2008, Nanotherapeutic particles: an emerging treatment modality for cancer, Nat Rev Drug Discov. 7(9):771-782, incorporated by reference. Long circulating macromolecular carriers, such as liposomes, can exploit the enhanced permeability and retention effect for preferential extravasation from tumor vessels. In certain embodiments, the complexes of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70. These cellular delivery systems may be introduced into the body transdermally through the methods described herein.

### iv. EXAMPLES

### Example vi(a): PEG

Example vi(a) includes embodiments of the invention that include a composition 101 for treating a viral infection, the composition comprising a programmable nuclease 107 linked to PEG. The use of PEG may aid the composition in avoiding immune clearance in a patient infected with a virus. Preferably the programmable nuclease is an RNA-guided nuclease such as a CRISPR-associated nuclease or Cpf1. In preferred embodiment, the RNA-guided nuclease has an amino acid sequence at least 90% similar to Cas9. The nuclease is present in ribonucleoprotein form with the nuclease complexed with a guide RNA, wherein a portion of the guide RNA is complementary to a target in a viral genome and not substantially complementary to any part of a human genome.

The PEG may be attached to the Cas9 at a side chain of an amino acid of the Cas9, wherein the side chain comprises an amine, a carboxyl, a sulfhydryl, or a carbonyl.

The PEG may be attached to the side chain through a linker, which may include a carbonyl, a disulfide bond, a thioether, an amine bond, a hydrazine linkage, an amide bond, an imidoester; maleimide.

### Example vi(b): Aptamers

Example vi(b) includes embodiments of the invention that include a composition for treating a viral infection, the composition comprising a programmable nuclease linked to an aptamer. Use of an aptamer may aid the composition in locating to, or remaining at, a specific target in a patient infected with a virus. For example, the aptamer may bind strongly and specifically to a viral protein. Preferably, the programmable nuclease is an RNA-guided nuclease such as a CRISPR-associated nuclease or Cpf1 (e.g., Cas9 or a nuclease with an amino acid sequence at least 90% similar to Cas9). In preferred embodiments of example vi(b), the nuclease is present in ribonucleoprotein form with the nuclease complexed with a guide RNA. A portion of the guide RNA is complementary to a target in a viral genome and not substantially complementary to any part of a human genome. The aptamer may be attached to the Cas9 at a side chain of an amino acid of the Cas9, wherein the side chain comprises an amine, a carboxyl, a sulfhydryl, or a carbonyl.

The secondary moiety is attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

### Example vi(c): apoE

Example vi(c) includes embodiments of the invention that include a composition for treating a viral infection, the composition comprising a programmable nuclease linked at least a portion of an apolipoprotein E (apoE) protein. The use of apoE may aid in delivering the composition across the blood-brain barrier of a patient infected with a virus. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(c), wherein the secondary moiety is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(c), wherein the nuclease and the secondary moiety are part of a fusion protein. The fusion protein may be expressed from a recombinant gene.

### Example vi(d): Fc Chain

Example vi(d) includes embodiments of the invention that include a composition for treating a viral infection, the composition comprising a programmable nuclease linked to an Fc region of an immunoglobulin. The use of Fc preferably aids the composition in avoiding immune clearance by the immune system of a patient infected by a virus. Because the Fc-nuclease fusion protein is not cleared by the infected patient's immune system, it remains and cleaves viral genetic material, thereby clearing the viral infection. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(d), wherein the secondary moiety is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(d), wherein the nuclease and the secondary moiety are part of a fusion protein. The fusion protein may be expressed from a recombinant gene.

### Example vi(e): Albumin

Example vi(e) includes embodiments of the invention that include a composition for treating a viral infection, the composition comprising a programmable nuclease linked to at least a portion of albumin. The use of albumin may significantly increase the half-life of the composition in vivo in a patient infected with a virus. Because the half-life of the composition is increased, the nuclease has more time to work (relative to an unmodified nuclease), and the viral infection is successfully cleared. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(e), wherein the secondary moiety is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(e), wherein the nuclease and the secondary moiety are part of a fusion protein. The fusion protein may be expressed from a recombinant gene.

### Example vi(f): Biotin

Example vi(f) includes embodiments of the invention that include a composition for treating a viral infection, the composition comprising a programmable nuclease linked to biotin, streptavidin, or avidin. Modification of a programmable nuclease with biotin, streptavidin, or avidin allows that nuclease to be easily linked strongly to some other moiety (e.g., any of the secondary moieties described herein may be linked to biotin, streptavidin, or avidin as a “tertiary moiety”). Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(f), biotin is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In some embodiments of example vi(f), streptavidin or avidin is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(f), the nuclease and biotin are part of a fusion protein. The fusion protein may be expressed from a recombinant gene (see FIG. 11).

### Example vi(g): A Lectin Protein

Example vi(g) includes embodiments of the invention that include a composition for treating a viral infection, the composition comprising a programmable nuclease linked to a lectin protein. Lectin may be linked to a programmable nuclease to target specific tissue such as the luminal surface of the small intestine, to trigger vesicular transport into or across epithelial cells, or both. Thus a lectin-modified programmable nuclease may be beneficial for specifically treating viral infections within epithelial cells of an infected patient.

Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

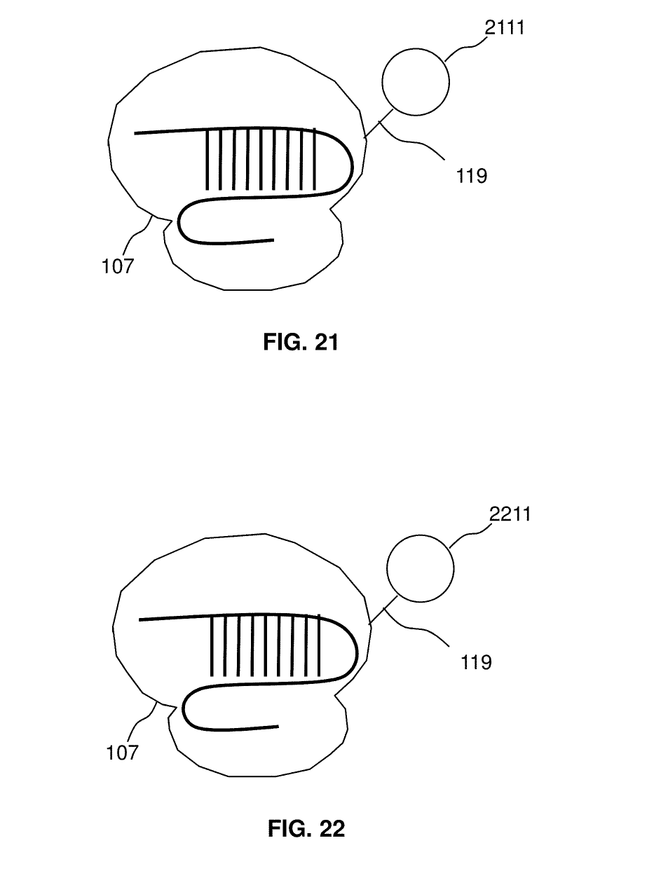

FIG. 21 shows a programmable nuclease 107 conjugated to a lectin protein 2111.

In some embodiments of example vi(g), wherein the lectin protein 2111 is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(g), the nuclease and the lectin protein are part of a fusion protein. The fusion protein may be expressed from a recombinant gene.

### Example vi(h): Carbohydrates

Example vi(h) includes embodiments of the invention that include a composition for treating a viral infection, the composition comprising a programmable nuclease linked to a carbohydrate/sugar. Carbohydrate-mediated delivery may be site specific/cell specific. Carbohydrates have been used for the delivery of macromolecular drugs. Lectins offer specific and noncovalent binding sites for defined carbohydrates. Specific delivery to liver cells may be aided by targeting cell surface lectins with specific carbohydrates conjugated to a programmable nuclease. Similarly, surface lectins of cancer cells participate provide a target specific to cancer cells. Thus, molecules such as lectins provide potential cell-specific targets for carbohydrate-modified programmable nucleases.

FIG. 22 shows a composition 2201 that includes a programmable nuclease 107 linked to a carbohydrate 2211. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments, carbohydrates are attached either to the amide nitrogen atom in the side chain of asparagine (termed an N-linkage) or to the oxygen atom in the side chain of serine or threonine (termed an O-linkage). Amino acid residues in Cas9 that may be linked to a carbohydrate include N14, N37, N46, N77, N88, N121, N175, N178, N193, N199, N202, N224, N235, N240, N251, N255, N277, N295, N309, N357, N394, N407, N436, N459, N477, N497, N501, N504, N522, N556, N588, N609, N611, N668, N690, N692, N726, N758, N767, N776, N803, N808, N818, N831, N854, N863, N869, N881, N888, N899, N940, N846, N980, N990, N1041, N1044, N1054, N1066, N1093, N1115, N1177, N1208, N1224, N1234, N1286, N1295, N1308, and N1317, to illustrate, as well as any serine or threonine, among others.

In some embodiments of example vi(h), wherein the sugar is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

### Example vi(i): Elastin

Example vi(i) includes embodiments of the invention that include a composition 2301 for treating a viral infection, the composition comprising a programmable nuclease 107 linked to at least a portion of an elastin protein 2311, optionally through a linker 819. It may be found that linking one or more elastin proteins to a nuclease increases target-site retention time of the therapeutic. The use of elastin may significantly increase the half-life of the composition in vivo in a patient infected with a virus. Because the half-life of the composition is increased, the nuclease has more time to work (relative to an unmodified nuclease), and the viral infection is successfully cleared.

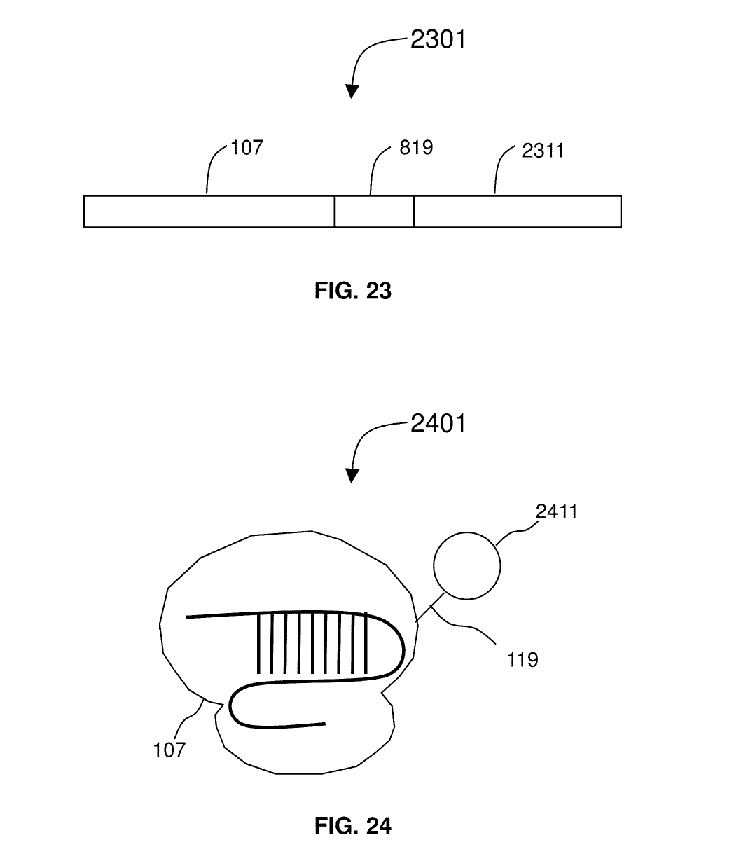

FIG. 23 shows the primary structure of include a composition 2301 that includes a programmable nuclease 107 linked to at least a portion of an elastin protein 2311 through a linker 819.

Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(i), wherein the elastin protein is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(i), the nuclease and the elastin protein are part of a fusion protein. The fusion protein may be expressed from a recombinant gene.

### Example vi(j): Cell-Penetrating Peptides

Cell-penetrating peptides (CPPs) are short peptides that facilitate cellular intake/uptake of various molecular equipment (from nanosize particles to small chemical molecules and large fragments of DNA). The “cargo” is associated with the peptides either through chemical linkage via covalent bonds or through non-covalent interactions. The function of the CPPs are to deliver the cargo into cells, a process that commonly occurs through endocytosis

Example vi(j) includes embodiments of the invention that include a composition for treating a viral infection, the composition comprising a programmable nuclease linked to a cell-penetrating peptide.

FIG. 24 shows a composition 2401 that includes a programmable nuclease 107 linked to a cell penetrating peptide 2411. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

The cell penetrating peptide 2411 will typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of cell-penetrating peptides are the hydrophobic peptides, containing only apolar residues, with low net charge or have hydrophobic amino acid groups that are crucial for cellular uptake. Sequences for cell-penetrating peptides are known in the literature and can be looked up online, for example at the web site cell-penetrating-peptides.org

In some embodiments of example vi(j), the cell-penetrating peptide is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(j), wherein the nuclease and the cell-penetrating peptide are part of a fusion protein. The fusion protein may be expressed from a recombinant gene.

### Example vi(k): Enzymes

Example vi(k) includes embodiments of the invention that include a composition for treating a viral infection, the composition comprising a programmable nuclease linked to an enzyme. Enzyme as used herein refers to a catalytically active polypeptide and thus includes catalytic domains of known enzymes, even where the complete polypeptide of the known enzyme is not used. An enzyme may be attached to a programmable nuclease in an antiviral therapeutic so that the enzyme performs its function in a patient infected with a virus.

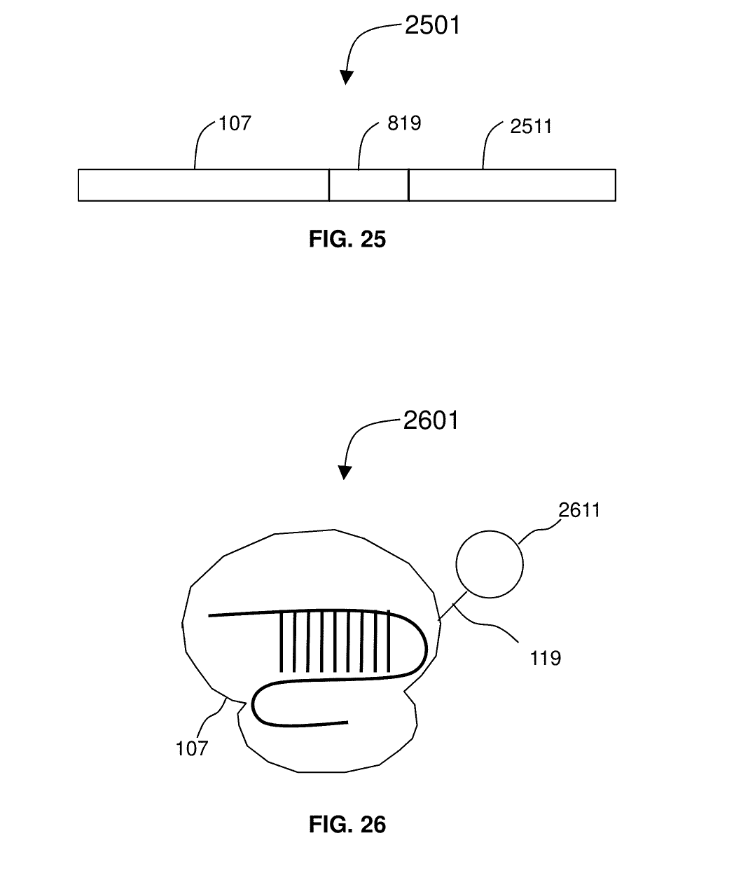

FIG. 25 shows a composition 2501 that includes a programmable nuclease connected to an enzyme 2511 through an optional linker 819. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

The enzyme 2511 may be any suitable enzyme. In a preferred embodiment, the enzyme 2511 may include at least a nuclease domain of FokI. In the described embodiment, the programmable nuclease 107 localizes to target viral genetic material by design. The FokI, which may otherwise cleave non-specifically, will then tend to cleave nucleic acid proximate to the viral genetic material. This may be particularly beneficial for removing viral episomes from cytoplasm. In particular, FokI cleavage may ensure fatal (to the virus) cleavage and prevent any hope of repair.

In some embodiments of example vi(k), wherein the enzyme is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(k), wherein the nuclease and the enzyme are part of a fusion protein. The fusion protein may be expressed from a recombinant gene.

### Example vi(l): Zinc Fingers

Example vi(1) includes embodiments of the invention that include a composition for treating a viral infection, the composition comprising a programmable nuclease linked to a zinc finger protein. A zinc finger may aid in localizing or targeting the programmable nuclease to viral genetic material. A zinc finger can be designed or included that binds to some specific target in the viral genome. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome. Where the nuclease is, e.g., Cas9, a zinc finger may be beneficial if the gRNA target presents binding challenges. For example, if the best available target for Cas9 binding tends to form hairpin structures, or has limited availability due to steric reasons, then including a zinc finger that binds to the viral genome a modest distance away from the Cas9 target can effectively seat the Cas9 near its intended target and improve its ultimate binding effectiveness and cleavage.

FIG. 26 shows a composition 2601 that includes a programmable nuclease 107 linked to a zinc finger 2611 through an optional linker 119.

In some embodiments of example vi(1), wherein the zinc finger protein is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(1), wherein the nuclease and the zinc finger protein are part of a fusion protein. The fusion protein may be expressed from a recombinant gene.

### Example vi(m): Epitopes

Example vi(m) includes embodiments of the invention that include a composition for treating a viral infection, the composition comprising a programmable nuclease linked to an antibody binding region. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(m), wherein the antibody binding region is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(m), wherein the nuclease and the antibody binding region are part of a fusion protein. The fusion protein may be expressed from a recombinant gene.

### Example vi(n): Protease Target

Example vi(n) includes embodiments of the invention that include a composition for treating a viral infection, the composition comprising a programmable nuclease linked to an enzyme cleave region (i.e., a polypeptide targeted by a protease). Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(n), wherein the enzyme cleave region is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(n), the nuclease and the enzyme cleavage region are part of a fusion protein. The fusion protein may be expressed from a recombinant gene.

### Example vi(o): Transcription Repressor

Example vi(o) includes embodiments of the invention that include a composition for treating a viral infection, the composition comprising a programmable nuclease linked to a transcription repressor. A transcription repressor may be linked to a programmable nuclease in an antiviral therapeutic to give the antiviral therapeutic a “one-two punch”. Not only does the nuclease cleave the viral genetic material, the transcription repressor will also bind and repress viral transcription, thus preventing any viral activity. Preferably the programmable nuclease is an RNA-guided nuclease such as a CRISPR-associated nuclease or Cpf1 (e.g., Cas9 or a modified Cas9 having an amino acid sequence at least 90% similar to Cas9). In some embodiments, the nuclease is present in ribonucleoprotein form with the nuclease complexed with a guide RNA. A portion of the guide RNA may be complementary to a target in a viral genome and not substantially complementary to any part of a human genome.

In some embodiments of example vi(o), the transcription repressor is attached to the Cas9 at a side chain of an amino acid of the Cas9, wherein the side chain comprises an amine, a carboxyl, a sulfhydryl, or a carbonyl. The secondary moiety may be attached to the side chain through a linker, which could include one or more of a disulfide bond, a thioether, an amine bond, a hydrazine linkage, an amide bond, an imidoester; maleimide; polyethylene glycol (PEG); BM(PEG)n or BS(PEG)n with 1<n<9; and biotin.

In certain embodiments of example vi(o), the nuclease and the transcription repressor are part of a fusion protein. The fusion protein may be expressed from a recombinant gene.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### APPENDIX

(SEQ ID NO: 1)
MDKKYSIGLD IGTNSVGWAV ITDEYKVPSK KFKVLGNTDR
HSIKKNLIGA LLFDSGETAE ATRLKRTARR RYTRRKNRIC
YLQEIFSNEM AKVDDSFFHR LEESFLVEED KKHERHPIFG
NIVDEVAYHE KYPTIYHLRK KLVDSTDKAD LRLIYLALAH
MIKFRGHFLI EGDLNPDNSD VDKLFIQLVQ TYNQLFEENP
INASGVDAKA ILSARLSKSR RLENLIAQLP GEKKNGLFGN
LIALSLGLTP NFKSNFDLAE DAKLQLSKDT YDDDLDNLLA
QIGDQYADLF LAAKNLSDAI LLSDILRVNT EITKAPLSAS
MIKRYDEHHQ DLTLLKALVR QQLPEKYKEI FFDQSKNGYA
GYIDGGASQE EFYKFIKPIL EKMDGTEELL VKLNREDLLR
KQRTFDNGSI PHQIHLGELH AILRRQEDFY PFLKDNREKI
EKILTFRIPY YVGPLARGNS RFAWMTRKSE ETITPWNFEE
VVDKGASAQS FIERMTNFDK NLPNEKVLPK HSLLYEYFTV
YNELTKVKYV TEGMRKPAFL SGEQKKAIVD LLFKTNRKVT
VKQLKEDYFK KIECFDSVEI SGVEDRFNAS LGTYHDLLKI
IKDKDFLDNE ENEDILEDIV LTLTLFEDRE MIEERLKTYA
HLFDDKVMKQ LKRRRYTGWG RLSRKLINGI RDKQSGKTIL
DFLKSDGFAN RNFMQLIHDD SLTFKEDIQK AQVSGQGDSL
HEHIANLAGS PAIKKGILQT VKVVDELVKV MGRHKPENIV
IEMARENQTT QKGQKNSRER MKRIEEGIKE LGSQILKEHP
VENTQLQNEK LYLYYLQNGR DMYVDQELDI NRLSDYDVDH
IVPQSFLKDD SIDNKVLTRS DKNRGKSDNV PSEEVVKKMK
NYWRQLLNAK LITQRKFDNL TKAERGGLSE LDKAGFIKRQ
LVETRQITKH VAQILDSRMN TKYDENDKLI REVKVITLKS
KLVSDFRKDF QFYKVREINN YHHAHDAYLN AVVGTALIKK
YPKLESEFVY GDYKVYDVRK MIAKSEQEIG KATAKYFFYS
NIMNFFKTEI TLANGEIRKR PLIETNGETG EIVWDKGRDF
ATVRKVLSMP QVNIVKKTEV QTGGFSKESI LPKRNSDKLI
ARKKDWDPKK YGGFDSPTVA YSVLVVAKVE KGKSKKLKSV
KELLGITIME RSSFEKNPID FLEAKGYKEV KKDLIIKLPK
YSLFELENGR KRMLASAGEL QKGNELALPS KYVNFLYLAS
HYEKLKGSPE DNEQKQLFVE QHKHYLDEII EQISEFSKRV
ILADANLDKV LSAYNKHRDK PIREQAENII HLFTLTNLGA
PAAFKYFDTT IDRKRYTSTK EVLDATLIHQ SITGLYETRI
DLSQLGGD

SEQ ID NO: 1 is the amino acid sequence of Cas9.

GGGS (SEQ ID NO: 2)

SEQ ID NO: 2 is a portion useful in a protein linker.

EAAAK (SEQ ID NO: 3).

SEQ ID NO: 3 is a portion useful in a protein linker.

What is claimed is:

1. A composition for treating a viral infection, the composition comprising a programmable nuclease linked to a secondary moiety.

2. The composition of claim 1, wherein the secondary moiety is linked to the programmable nuclease at a side chain of an amino acid of the programmable nuclease.

3. The composition of claim 2, wherein the programmable nuclease comprises an RNA-guided nuclease and the secondary moiety is selected from the group consisting of polyethylene glycol (PEG); an aptamer; at least a portion of an apolipoprotein E (apoE) protein; at least a portion of an Fc region of an immunoglobulin (Fc-domain); and at least a portion of albumin.

4. The composition of claim 3, wherein the programmable nuclease is present as ribonucleoprotein in which the RNA-guided nuclease is complexed with a guide RNA, wherein a portion of the guide RNA is complementary to a target in a viral genome and not substantially complementary to any part of a human genome.

5. The composition of claim 4, wherein the secondary moiety is attached to the side chain through a linker.

6. The composition of claim 5, wherein the linker comprises one selected from the group consisting of a disulfide bond; a thioether; an amine bond; a hydrazine linkage; an amide bond; an imidoester; a peptide bond; maleimide; a click reaction product; one or more five-membered heterocycles; polyethylene glycol (PEG); BM(PEG)n with 1<n<9; poly lactic-co-glycolic acid (PLGA)-b-PEG; and biotin.

7. The composition of claim 4, wherein the RNA-guided nuclease has an amino acid sequence at least 90% similar to Cas9.

8. The composition of claim 2, wherein the nuclease is present as deoxyribonucleoprotein (DNP).

9. The composition of claim 8, wherein the DNP comprises an NgAgo protein in complex with guide DNA (gDNA) that is complementary to a target in a viral genome and not substantially complementary to any part of a human genome, and the secondary moiety is selected from the group consisting of polyethylene glycol (PEG); an aptamer; at least a portion of an apolipoprotein E (apoE) protein; at least a portion of an Fc region of an immunoglobulin (Fc-domain); and at least a portion of albumin.

10. The composition of claim 8, wherein the DNP comprises a DNA-guided nuclease having an amino acid sequence at least 90% similar to NgAgo.

11. The composition of claim 2, wherein the programmable nuclease comprises a TALEN protein engineered to recognize a target in a viral genome but not recognize the human genome and the secondary moiety is selected from the group consisting of polyethylene glycol (PEG); an aptamer; at least a portion of an apolipoprotein E (apoE) protein; at least a portion of an Fc region of an immunoglobulin (Fc-domain); and at least a portion of albumin.

12. The composition of claim 1, wherein the secondary moiety and the programmable nuclease are both part of a recombinant protein.

13. The composition of claim 12, wherein the programmable nuclease is one selected form the list consisting of: a TALEN protein engineered to recognize a target in a viral genome but not recognize the human genome; a DNA-guided nuclease; and an RNA-guided nuclease.

14. The composition of claim 13, wherein the secondary moiety is selected from the group consisting of at least a portion of an apolipoprotein E (apoE) protein; at least a portion of an Fc region of an immunoglobulin; at least a portion of albumin; and biotin.

15. The composition of claim 14, wherein the recombinant protein includes a linker between the secondary moiety and the programmable nuclease.

16. The composition of claim 12, wherein the programmable nuclease is an RNA-guided nuclease and the RNA-guided nuclease is present in ribonucleoprotein form with the nuclease complexed with a guide RNA, wherein a portion of the guide RNA is complementary to a target in a viral genome and not substantially complementary to any part of a human genome.

17. The composition of claim 16, wherein the secondary moiety is selected from the group consisting of at least a portion of an apolipoprotein E (apoE) protein; at least a portion of an Fc region of an immunoglobulin; at least a portion of albumin; and biotin.

18. The composition of claim 17, wherein the RNA-guided nuclease has an amino acid sequence at least 90% similar to Cas9.

19. The composition of claim 15, wherein the linker comprises a plurality of glycine residues.

20. The composition of claim 1, wherein the secondary moiety is non-covalently bound to the programmable nuclease.

21. The composition of claim 20, wherein the secondary moiety is non-covalently bound to the programmable nuclease through a biotin/streptavidin linkage.

22. The composition of claim 1, wherein the nuclease is covalently linked to the secondary moiety

23. The composition of claim 1, wherein the nuclease is linked to the secondary moiety through a linker

24. The composition of claim 23, wherein the linker comprises protein.

25. The composition of claim 24, wherein the linker comprises at least one selected from the group consisting of: a plurality of proline residues; a plurality of glycine residues; and a plurality of threonine and serine residues.

26. The composition of claim 23, wherein the linker comprises a non-protein chemical linker.

27. The composition of claim 26, wherein the non-protein chemical linker comprises one selected from the group consisting of: a disulfide bond; a thioether bond; an amine bond; a hydrazine linkage; an amide bond; an imidoester; maleimide; PEG; and BM(PEG)n with 1<n<9.

28. The composition of claim 23, wherein the linker is attached to the programmable nuclease at an amino acid in the nuclease selected from the group consisting lysine, cysteine, aspartic acid, and glutamic acid.

29. The composition of claim 23, wherein the linker is biodegradable.

30. The composition of claim 23, wherein the linker is cleavable.

31. The composition of claim 30, wherein the linker comprises a target of a protease.

32. The composition of claim 1, wherein the secondary moiety is selected from the group consisting of: polyethylene glycol (PEG); an aptamer; at least a portion of an apolipoprotein E (apoE) protein; an Fc region of an immunoglobulin; at least a portion of albumin; biotin; streptavidin; avidin; a lectin protein; a sugar; at least a portion of an elastin protein; a cell-penetrating peptide; an enzyme; a nuclease domain of FokI; a zinc finger protein; an antibody binding region; a target of a protease; a transcription repressor; at least a portion of an antibody non-covalently bound to the programmable nuclease; and an aptamer non-covalently bound to the programmable nuclease.

33. The composition of claim 1, wherein the programmable nuclease is an RNA-guided nuclease.

34. The composition of claim 33, wherein the RNA-guided nuclease is a CRISPR-associated nuclease or Cpf1.

35. The composition of claim 1, wherein the nuclease comprises at least one substitution relative to a naturally-occurring version of the nuclease.

36. The composition of claim 35, wherein the at least substitutions reduce intracellular protein binding of the nuclease or reduces off-target toxicity of the composition.

37. The composition of claim 1, wherein the nuclease is a Cas9 protein encoded by an mRNA that is co-delivered with gRNA.

38. The composition of claim 1, wherein the nuclease is a NgAgo protein encoded by an mRNA that is co-delivered with gDNA.

39. The composition of claim 1, wherein the nuclease is a TALEN encoded by an mRNA.


SEQUENCE LISTING (3 sequences)

SEQ ID NO: 1 - protein, 1368 aa, Streptococcus pyogenes [sequence omitted]

SEQ ID NO: 2 - protein, 4 aa, Unknown
  Short protein linker having no organism associated.
GGGS

SEQ ID NO: 3 - protein, 5 aa, Unknown
  Short protein linker without an associated organism.
EAAAK